(19) United States
(12) Patent Application Publication
(10) Pub. No.: US 2021/0087533 A1
(43) Pub. Date: Mar. 25, 2021

(54) CANINE EPITHELIAL ORGANOIDS AND METHODS OF MAKING, RECOVERING, AND USE

(71) Applicant: Iowa State University Research Foundation, Inc., Ames, IA (US)

(72) Inventors: Jonathan P. Mochel, Ames, IA (US); Karin Allenspach Jorn, Ames, IA (US); Albert E. Jergens, Ames, IA (US); Todd Alan Atherly, Sarasota, FL (US)

(21) Appl. No.: 16/948,450

(22) Filed: Sep. 18, 2020

Related U.S. Application Data
(60) Provisional application No. 63/003,342, filed on Apr. 1, 2020.
(60) Provisional application No. 62/902,833, filed on Sep. 19, 2019.

(51) Int. Cl. C12N 5/071

(52) U.S. Cl. CPC C12N 5/0679; C12N 2513/00; C12N 2501/119; C12N 5/0697

(57) ABSTRACT

The present invention relates to organoids of canine epithelial tissues and organs. Methods for culturing, freezing, and recovering of the epithelial organoids as well as using the epithelial organoid for detection of changes due to disease, environmental or dietary triggers, and drug screening and testing for safety for efficacy and safety and personalized medicine are also included.

### CROSS-REFERENCE TO RELATED APPLICATIONS

This application priority under 35 U.S.C. § 119 to Provisional Application U.S. Ser. No. 62/902,833 filed on Sep. 19, 2019 and Provisional Application U.S. Ser. No. 63/003,342 filed on Apr. 1, 2020, both of which are herein incorporated by reference in their entireties.

### GOVERNMENT RIGHTS CLAUSE

This invention was made with Government support under the National Science Foundation, contract Grant Number IIP1912498. The Government has certain rights in the invention.

### FIELD OF THE INVENTION

The present invention relates to compositions for the growth of canine epithelial organoids. More specifically, the compositions include models for the study of developmental biology of the intestines, stomach, liver, bladder, and other epithelial tissues; drug discovery and toxicity screening; drug testing for personalized medicine; infectious disease biology of viruses; bacteria and other infectious agents; the interaction of the microbiome with the epithelial cell layer; cancer; regenerative and personalized medicine. Methods and systems for culturing, freezing, and recovering of the frozen cells are also provided as are methods for employing the compositions.

### BACKGROUND OF THE INVENTION

Rodent models, especially the mouse, have been extensively used to study gastrointestinal (GI) diseases due to cost effectiveness, ethical considerations, and the easy accessibility to genetically engineered technology. Despite the wide use of mouse models in biomedical research, the translational value of mouse studies for human disease remains controversial. In addition, mice and other rodents often fail to adequately represent the human condition, as well as drug response in toxicity and efficacy studies. Given the high failure rate of drugs from discovery and development through the clinical trial phase (i.e., more than 90%), there is now a critical need for better animal models for preclinical studies.

Large animal models, such as the dog, are typically more representative than mice as they have a relatively large body size, longer life span, more closely resemble human GI physiology, and develop spontaneous, analogous diseases including inflammatory bowel disease (IBD) and colorectal cancer (CRC). Dogs have been used as an animal model for human health and disease from the ancient to the modern era. The dog is still considered to be superior to non-rodent mammalian animal models for pharmaceutical research and is preferred by the FDA for initial safety data of drugs for human use. Although the dog has contributed immensely to the advancement of medical knowledge in the past, the use of the dog in medical research has declined in recent years due to the emotional perceptions among the public and ensuing ethical concerns with canine research.

However, there is currently a limited number of canine-specific primary cell lines to investigate epithelial physiology, such as intestinal physiology, ex vivo or in vitro. For example, for intestinal physiology the well-characterized immortalized cell lines including the Madin-Darby canine kidney (MDCK) cells do not accurately model intestinal epithelial interactions in the dog due to their origin from immature kidney cells. Recently, isolated primary canine intestinal epithelial cells have been immortalized with a temperature-sensitive mutant of the Simian Virus 40 large tumor antigen (SV40 T-Ag). Although this cell line can be grown on a monolayer, the SV40 T-Ag may initiate pathways which could provide spurious, non-physiologic findings ex vivo given its tumorigenic cell line origin.

The canine GI organoids arose as a model to bridge the gap in the drug development pipeline by providing a more representative in vitro model to test drug efficacy and toxicity in preclinical studies, as well as an innovative screening tool in drug discovery, while also reducing the number of animals needed for in vivo studies. Thus, the ultimate goal of our research is to culture canine intestinal organoids from healthy and diseased dogs to develop better therapeutic strategies and personalized medicine for both animal and human health.

Stem cell-derived 3D organoids have emerged as a cutting-edge cell culture technology to study the developmental biology of the intestines, brain, stomach, and liver; drug discovery and toxicity screening; drug testing for personalized medicine; infectious disease biology of the microbiome, including bacteria and viruses; and regenerative medicine. Organoids are collections of organ-specific cell aggregates derived from either primary tissue or stem cells that are capable of organ-like functionality in an in vitro environment. The 3D organoid model better reproduces the in vivo biology, structure, and function, as well as genetic and epigenetic signatures of original tissues, unlike widely used two-dimensional (2D) cell monolayer models that utilize cancer and immortalized cell lines.

Organoids may be developed from either embryonic or induced pluripotent derived stem cells (iPSC) or organ-specific adult stem cells (ASC). Organoids derived from ASCs are generated without genetic transduction by transcription factors, unlike organoids derived from iPSCs, thus providing a more physiologically relevant in vitro model than iPSC-derived organoids. ASC-derived organoids are a functional model that can be differentiated to replicate the in vivo adult environment and can be safely transplanted into animals and humans. In addition, adult intestinal stem cell (ISC)-derived organoids have recently gained attention as a model to understand how the intestinal epithelia interact with the gut microbiome to modulate GI health and disease, for the study of infectious diseases of the GI tract, and as a drug screening tool for personalized medicine in diseases such as cystic fibrosis (CF).

Traditional in vitro methods rely on 2D cell culture methods. However, these cell culture methods have several disadvantages, such as a low survival rate of primary epithelial cells in culture, ontogenetically transformed cell lines have poor translational value, and 2D monolayer cultures are limited in reproducing physiology. For example, the most common system to evaluate transport of drugs through intestine is based on human colon cancer-derived Caco-2 cells. There are several limitations to this model, including the inconsistent expression of certain key cellular proteins (such as tight junction proteins, which are especially important for intestinal permeability), compared to that observed in vivo in enterocytes, as well as significant lab-to-lab variability in the experimental results. Further 2D cell cultures lack the complex 3D structures common in organs and are typically just a single cell type, unlike what is found in organs.

However, depending on the use, there may be limitations to the 3D intestinal organoid systems. For instance, the 3D organoid body prevents ready access to the lumen for studying the interactions with dietary constituents, microorganisms, drugs, or environmental or dietary triggers transported through an epithelial layer. While microinjection of a luminal component (e.g., living bacterial cells or other products) into the lumen of an organoid has been feasible, the technique can be challenging due to the heterogeneity in organoid size, adverse effects of the injection, and the requirement of specialized techniques and equipment. Thus, 3D cultures of a polarized intestinal cell monolayer are better suited for the standardized measurement of transepithelial permeability and epithelial-luminal interaction due to easier accessibility to the apical surface. Moreover, creating a canine-derived intestinal interface may be further improved by integrating the optimized protocol to the intestinal microphysiological systems.

It is therefore an object of this disclosure to provide intestinal stem cell derived models for studying canine epithelia tissue. For example, testing P-glycoprotein (P-gp) transport for the study of drug absorption in dogs or testing the efficacy and toxicity of a chemotherapeutic for personalized medicine. It is also an object of the disclosure to provide methods of making, freezing, and recovering of epithelial organoids. It is also a further objective to provide methods for using genetically-modified organoids for regenerative or personalized medicine.

It is a further object of the disclosure to provide methods of using the models for testing drugs and performing epithelial research, such as diseases like inflammatory bowel disease and cancer.

It is another object of the disclosure to provide systems using the models for drug testing and screening and for the studying of epithelial physiology, both in healthy and diseased states, and in different environmental or dietary regimes. These studies may lead to the use of the canine epithelial organoids for personalized medicine.

Other objects, aspects and advantages of this invention will be apparent to one skilled in the art in view of the following disclosure, the drawings, and the appended claims.

### SUMMARY OF THE INVENTION

An advantage of the invention is to provide models which more closely reflect the physiological state of a subject or subjects than the currently available model systems. It is an advantage of the present invention that the models may be further genetically-modified. It is also an advantage of the models that they either represent a single time point or by taking advantage of the shorter lifespan of canines compared humans to be create longitudinal canine models for chronic human diseases. It is a further advantage of the models that both healthy and diseased models may be made from the same animal.

In an embodiment, the present invention provides stem cell derived organoid models. The stem cells are grown in media that first allows their differentiation into their complex 3D structures and then a media that promotes growth. In an embodiment, the organoids are spherical and grown in solution. In a further embodiment, the cells are grown in an extracellular matrix. In another embodiment, the organoids are grown flat on a membrane or plate to provide ready access to the lumen of the organoid. In other embodiments, the intestinal stem cell derived model is a two-dimensional monolayer of an organoid grown on a permeable membrane, such as, but not limited to, a TRANSWELL® membrane. In another embodiment are methods for growing the organoids in either spherical form. In still yet another embodiment are methods for growing the organoid on a substrate.

In an embodiment, the present invention provides adult stem cell derived organoid models for physiological and disease research. In a further embodiment, a healthy control is compared to a diseased sample. In a further embodiment, the healthy control originates from the same animal as the diseased sample. In another embodiment, the healthy sample is derived from a different animal than the diseased sample. In an embodiment, the disease is cancer or inflammatory bowel disease.

In an embodiment, the present invention provides adult stem cell derived organoid models for testing drug absorption, efficacy, and safety. In a further embodiment, the model uses P-glycoprotein (P-gp) transport to study drug absorption. In some embodiments, the stem cells are derived from control or healthy subjects. In other embodiments, the stem cells are derived from subjects with a disease or which have been genetically-modified. Models made from control or healthy subjects may be used to test and screen drugs for normal physiological absorption while organoids derived from diseased or genetically-modified subjects may be used to test and screen drugs under various physiological conditions.

In an embodiment, the organoid models and methods of use described herein provide three-dimensional culture conditions, including passaging, freezing, and recovery of the frozen organoids. These models may be used for screening of potential therapeutic drugs and screening of drug responses in ex vivo models. The embodiments provide a canine-specific system for testing P-gp affinity in in therapeutic drug development. As referred to herein, drug screening and development can include pharmacotherapeutic effects, bioavailability, elimination, efficacy, and various safety effects, among others. In an embodiment, the organoids are able to predict clinical responses, such as efficacy and/or adverse effects, and thereby enable designing therapies, including therapies for healthy subjects, diseased subjects, and/or any subject requiring personalized treatment. These embodiments include the optimization of individualized medicine, and testing of the bioavailability of drugs across the intestinal tract.

In various embodiments, the drug may be administered to a subject orally, intravascularly (IV), intramuscularly (IM), subcutaneously (SC), or intraperitoneally (IP). In a preferred embodiment, the drug is administered orally. Drugs delivered via non-oral routes may still undergo P-gp transport in other organs, such as, but not limited to, the liver (biliary excretion), the kidneys or the blood brain barrier, and so the models may be used to screen drugs which may be transported in non-intestinal organs.

In some embodiments the drug is fluorescent. In other embodiments the drug is conjugated with a reporter.

In an embodiment, the organoid is derived from epithelial tissue. In some embodiments the organoid is an enteroid derived from the small intestine. In some embodiments the enteroid is derived from the duodenum or jejunum. In other embodiments, the enteroid is derived from the ileum. In yet other embodiments the intestinally derived organoid is a colonoid derived from the large intestine. In still other embodiment, the organoid is derived from urothelial cells.

In an embodiment, the models include a compound which interacts with P-gp. In an embodiment the compound is an inhibitor. In another embodiment the compound is an inducer. In yet other embodiments the compound is a substrate.

In an embodiment, the intestinal stem cell derived models are genetically-modified after the stem cells have been purified. In other embodiments, the subject from which the stem cells are obtained is genetically-modified. In yet other embodiments, the subject from which the stem cells are obtained is diseased.

In an embodiment, the model represents a single time point. In another embodiment, the model is a longitudinal model where stem cells have been extracted from the same subject over time.

In an embodiment the methods include administering to a model a drug and a P-gp interacting compound; measuring the rate of transport of the drug across P-gp; and comparing the rate of transport to a model lacking the P-gp interacting compound. If said drug is a substrate for P-gp, then the P-gp interacting compound is preferably an inhibitor to control for the effect on transport of P-gp. If the drug is an inhibitor or inducer of P-gp, then the P-gp interacting compound is preferably a P-gp substrate in order to measure the effects of the drug on the transport function of P-gp. In a further embodiments, additional inhibitors, inducers, or substrates may be administers.

In another embodiment the present invention includes systems using the models to test or screen a drug for P-glycoprotein transport comprising the model of the invention, a P-gp interacting compound; and a way of detecting the transportation. In some embodiments the way of detecting the transportation is a change in fluorescence. In other embodiments the way of detecting the transportation may be a binding assay, such as an antibody detection system. In other embodiments the way of detecting the transportation may be through high performance liquid chromatography (HPLC) and mass spectrometry (MS). In still other embodiments, detection may be through staining. While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1A shows the localization of P-glycoprotein (P-gp) proteins in ileal tissue biopsies (arrow heads). FIG. 1B shows the localization of the P-gp proteins in ileal enteroids (arrow heads) from the same dogs as FIG. 1A revealed by immunohistochemistry staining (Discovery Ultra, Ventana Medical Systems, Inc.). Representative pictures from at least N=10 tissue sections and N=20 enteroids from a dog (total number of dogs=10). Scale bar: 100 μm.

FIG. 2 shows the protein expression levels of P-gp in ileal enteroids compared to original intestinal biopsy tissues in box whisker plot with data generated from at least N=10 tissue sections and N=20 ileal organoids derived from each dog (total number of dogs=8).

FIG. 3A shows a representative confocal image of P-gp function in canine enteroids. P-gp function was determined by incubation with Rh123, a P-gp substrate, at 1 μM, 10 μM, 20 μM and 50 μM with or without 20 μM of verapamil, a P-gp inhibitor, for 30 minutes. Enteroids were approximately 100 μm in size. Representative pictures of at least N=20 enteroids per treatment in duplicate. FIG. 3B shows the means and the standard deviations with 95% confidence internals were plotted for treatment groups without verapamil (white box) and with 20 μM verapamil (black box). Significant slope difference between CNT vs TNT was determined by a linear regression analysis (P<0.01) [β for black box line, (with verapamil)=X mean luminal intensity/Rh123 concentration. 13 for white box line, (without verapamil)=Y mean luminal intensity/Rh123 concentration]. Significant inhibition of the luminal transport of Rh123 with 20 μM of verapamil was noted at 1 μM (P<0.01), 10 μM (P<0.01), 20 μM (P<0.01), and 50 μM (P<0.05) of Rh123. (*P<0.05, ** P<0.01).

FIG. 4 shows a stable transfection of canine intestinal enteroids by CRISPR/Cas9 to knockout MDR1 gene or GFP control plasmid.

FIG. 5 shows three independent lines of canine colonoids show similar profile of epithelial barrier function when those three lines were used to form a monolayer on a nanoporous insert. The result was produced with 2 biological replicates, where each biological replicate was performed with 4 technical replicates. Error bars indicate SEM.

FIG. 6A shows a canine colonoid-derived monolayer on Day 13 was used to visualize the markers highlighting the cell lineages, proliferation, and mucus production of stem cells Lgr5+, Yellow). FIG. 6B the canine colonoid-derived monolayer on Day 13 was used to visualize the markers highlighting the cell lineages, proliferation, and mucus production of proliferative cells (Ki67, Red). FIG. 6C shows the canine colonoid-derived monolayer on Day 13 was used to visualize the markers highlighting the cell lineages, proliferation, and mucus production of absorptive enterocytes (ALPI, Magenta), FIG. 6D shows the canine colonoid-derived monolayer on Day 13 was used to visualize the markers highlighting the cell lineages, proliferation, and mucus production of enteroendocrine cells (Neurog3, Red). FIG. 6E shows the canine colonoid-derived monolayer on Day 13 was used to visualize the markers highlighting the cell lineages, proliferation, and mucus production of enteroendocrine cells (CgA, Red). FIGS. 6A, 6C and 6D were visualized by using RNA in situ hybridization and FIGS. 6B and 6E were visualized using IF staining. As a counterstaining, E-cadherin (Cyan for FIGS. 6A, 6C, and 6D), F-actin (Green for FIG. 6B and Cyan for FIG. 6E), or nuclei (Grey for FIGS. 6A, 6B, 6C, 6D, and 6E) were displayed. Bars, 20 μm. FIG. 6F shows the quantification of the population of the cells that show positive signals to the target markers normalized by the total numbers of nuclei. Three independent fields of view from two or more independent biological replicates were used. In each biological replicate, 2 technical replicates were performed. Error bars indicate SEM.

FIG. 7A shows the expression of P-gp being visually characterized by IF staining in an angled (upper) and cross-sectional side views (lower) show the localization the P-gp proteins (Yellow) on the polarized colonoid-derived monolayer at day 3. FIG. 7B shows the expression of P-gp being visually characterized by IF staining in an angled (upper) and cross-sectional side views (lower) show the localization the P-gp proteins (Yellow) on the polarized colonoid-derived monolayer at day 13. Nuclei, Cyan. Dashed lines pinpoint the location of the basement membrane in the nanoporous insert. Bars, 50 μm. FIG. 7C shows the quantification of the P-gp expression at days 3 and 13, respectively. Total 10 randomly chosen fields of view were used to detect P-gp expression levels among 4 biological replicates. In each biological replicate, we performed 2 technical replicates. a.u., arbitrary unit. Error bars indicate SEM.*P<0.0001.

FIG. 8 shows the quantification of the expression level of ZO-1 and E-cadherin at days 3 and 13 was performed using total 10 and 6 randomly chosen fields of view for ZO-1 and E-cadherin, respectively, among 4 biological replicates of IF staining experiment. We also applied two technical replicates to individual biological replicates. a.u., arbitrary unit. NS, not significant.

FIG. 9A shows a 3-Plex Positive Control Probe (Advanced Cell Diagnostics) was applied to the canine monolayer cultured for 13 days to confirm the functionality of the kit applied for a low expressor RNA (RNA Polymerase II Subunit A (POLR2A), Opal 650. FIG. 9B shows a 3-Plex Positive Control Probe (Advanced Cell Diagnostics) was applied to the canine monolayer cultured for 13 days to confirm the functionality of the kit applied for a high expressor RNA (Ubiquitin C (UBC), Opal 520. FIG. 9C shows an overlaid image is displayed in FIG. 9A. Nuclei, blue. Bars, 50 μm.

FIG. 10A shows a growth profile of the colonoid isolated from the canine colonic crypt. A small spherical colonoids progressively grows to form fully grown colonoids. Representative phase-contrast micrographs were taken at days 1, 3, and 5. The zoomed-in inset at each day shows the high-power magnification of a colonoid in the white dashed box. FIG. 10B shows a schematic displays the procedure of the formation of an epithelial monolayer derived from 3D canine colonoids. The fully-grown organoids are dissociated into single cells, then seeded into a nanoporous insert to form a monolayer. AP, apical; BL, basolateral. FIG. 10C shows a representative phase-contrast micrograph on day 3 and 13 are provided, respectively. Bars, 200 μm.

FIG. 11A shows a low magnification SEM image of the microvilli on the apical cell surface. FIG. 11B shows a high-power magnification of the microvilli from A indicated by a white dashed box. Bars, 5 μm. FIG. 11C shows a TEM image of the microvilli on the cell monolayer. MV, microvilli. Bar, 500 nm. FIG. 11D shows a high-power TEM image that shows the microvilli (MV) and the surrounding glycocalyx (GLX). Bar, 200 nm.

FIG. 12A shows a representation of mucus production (WGA) as visualized by live-cell imaging at the apical surface of the monolayer. Bar, 20 μm. FIG. 12B shows a representative TEM image shows the goblet cell with multiple mucin granules (MG) and mitochondria (M). Bar, 1 μm. FIG. 12C shows a low magnification SEM image of a goblet cell on the apical cell surface of the canine colonoid-derived monolayer. Bar, 5 μm.

FIG. 12D shows a high magnification of a goblet cell orifice (GO), a fenestrated membrane (FM) extending deep into the cell, and microvilli (MV) from C indicated by a white dashed box. Bar, 1 μm.

FIG. 13A shows a visualization of the spatial localization of the ZO-1 (Magenta) on the same location of a canine colonoid-derived monolayer as FIG. 10B. FIG. 13B shows a visualization of the spatial localization of E-cadherin (Cyan) on the same location of a canine colonoid-derived monolayer as FIG. 10A. Nuclei, Grey. Bar, 50 μm. FIG. 13C shows a profile of the epithelial barrier function was monitored by measuring TEER. The effect of culture medium on TEER was demonstrated by applying the regular proliferation medium in both the apical and basolateral side of the TRANSWELL® (Control, open circle) versus the differentiation/proliferation medium in the apical/basolateral compartments, respectively (Diff; closed circle). Both groups were cultured with the proliferation medium by Day 4 (a dashed line), then different culture media were applied (Diff vs. Control) for additional 4 days. Two biological replicates with 4 technical replicates were used in each condition. *P<0.01. FIG. 13D shows a TEM image of the intercellular junctional complex in the canine colonoid-derived monolayer. FIG. 13E shows a zoom-in of FIG. 10D that shows a high-power magnification of the white dashed area in FIG. 10D. MV, microvilli; M, mitochondria; and D, desmosome. Bars, 500 nm. FIG. 13F shows a profile of TEER (open circle) and corresponding apparent permeability (Pₐₚₚ) of fluorescein (closed square) on the days of 2, 3, 5, and 6 of the cultures. Each data point was prepared with 2 biological and 4 technical replicates. Error bars indicate SEM.

FIG. 14 shows a representation of the shared histological appearance and overexpression of CD44, FOXA1, and KT-7 gene transcripts.

FIG. 15 shows a graphical representation of Reduced metabolic activity of UC organoids following period of incubation with chemotherapeutics for 24-48 hours.

FIG. 16 shows a representation of canine ileal organoids from healthy dogs and dogs with IBD. Representative images of differentiated 5-7-day-old ileal enteroid were obtained with Leica Application Suite (LAS) software at x 40 magnification. Scale bar: 50 μm.

FIG. 17A shows a representative image of RNA-ISH and IHC at x 40 magnification. Scale bar: 50 μm. NeuroG3 and PAS expression in organoids shows significant difference between healthy and IBD organoids. Expression of ZO-1 exhibits similar trend between IBD organoids and tissues. FIG. 17B shows a graphical representation of the expression of NeuroG3 in organoids. FIG. 17C shows a graphical representation of the expression of PAS in the organoids. FIG. 17D shows a graphical representation of the expression of ZO-1 in the organoids. FIG. 17E shows a graphical representation of the expression of ZO-1 in the tissue extracts.

FIG. 18A shows a representative example of Forskolin induces swelling of IBD enteroids indicating presence of functional CFTR-Cl-channels. Representative images of stimulated enteroids were taken after 0 and 1 hr at x 5 magnification. Scale bar: 500 μm. Histogram of mean enteroid area from n=12 fields per condition (Control vs. Forskolin) as measured by ImageJ. FIG. 18B shows a graphical representation of Forskolin induces swelling of IBD enteroids.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE PREFERRED EMBODIMENTS

The present invention relates to methods and compositions for the growth of intestinal organoids for the study of oral drug P-glycoprotein (P-gp) mediated absorption in dogs. The embodiments are not limited to particular models, methods of making the models, using the models for drug testing or screening, and compositions, which can vary and are understood by skilled artisans.

It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers within the defined range. Throughout this disclosure, various aspects are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range (e.g. 1 to 5 includes 1, 1.5, 2, 2.75, 3, 3.80, 4, and 5).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. Whether or not modified by the term “about”, the claims include equivalents to the quantities. The term “actives” or “percent actives” or “percent by weight actives” or “actives concentration” are used interchangeably herein and refers to the concentration of those ingredients involved in cleaning expressed as a percentage minus inert ingredients such as water or salts.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

As used herein “organoids” refer to ex vivo models that are grown from adult stem cells to provide structures that resemble an organ in culture.

As used herein, the term “basal media” refers to a culture media that lacks some supplements that may be required for cell growth.

As used herein, the term “complete media” refers to a culture media that contains all the supplements to supports cell growth.

As used herein, the term “differentiation media” means any media that induces a stem cell, for example an induced pluripotent stem cell or an adult derived stem cell, to differentiate into the desired epithelial cells comprising the organoids.

As used herein the term “protecting media” refers to a differentiation media which inhibits cell death during cell culture.

As used herein, the term “freezing media” means any media in which the organoids may be frozen in and then recovered.

As used herein, the term “P-glycoprotein interacting compound” or “P-gp interacting compound” is any compound that functions as an inhibitor, inducer, or substrate for P-gp. An inhibitor may reduce the transport ability of P-gp, an inducer may increase the transport of P-gp, and a substrate may be transported by P-gp.

As used herein “antibodies” and like terms refer to immunoglobulin molecules and immunologically active portions of immunoglobulin (Ig) molecules, i.e., molecules that contain an antigen binding site that specifically binds (immunologically reacts with) an antigen. These include, but are not limited to, polyclonal, monoclonal, chimeric, single chain, Fc, Fab, Fab′, and Fab₂ fragments, and a Fab expression library. Antibody molecules relate to any of the classes IgG, IgM, IgA, IgE, IgD, which differ from one another by the nature of heavy chain present in the molecule. These include subclasses as well, such as IgG1, IgG2, and others. The light chain may be a kappa chain or a lambda chain. Reference herein to antibodies includes a reference to all classes, subclasses, and types. Also included are chimeric antibodies, for example, monoclonal antibodies or fragments thereof that are specific to more than one source, e.g., a mouse or human sequence.

The term “pharmaceutical agent” or “drug” refers to a chemical compound or composition capable of inducing a desired therapeutic or prophylactic effect when properly administered to a subject.

The term “sample” as referred to herein means an isolated part of an animal. Samples can include, but are not limited to, tissue sections, stem cells, cancerous cells, and tissue biopsies.

The term “subject” as used herein refer to a human or mammalian animal. The mammalian animal may include carnivores/omnivores or herbivores. Carnivores/omnivores may include canines, pigs, rodents, or felines.

The term “substantially free” as used herein refers to the amount of a compound may be present in a composition in so low as to not have a measurable effect. It should be noted that the compound may be present in the composition, for example, a specific growth factor is not added to a differentiation media may still be present in an organoid culture due to the organoid itself producing the growth factor.

The methods, compositions, and systems may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

The methods, compositions, and systems may be substantially or essentially free of components and ingredients. As used herein, “substantially free” and “essentially free” mean that a component or ingredient may be present in the methods, compositions, or systems, but do not contribute any property to the methods, compositions, or systems.

### 3D Organoid

The 3D Organoid model better reproduces the in vivo biology, structure, and function, as well as genetic and epigenetic signatures of original tissues, unlike widely used two-dimensional (2D) cell monolayer models that utilize cancer and immortalized cell lines.

Organoids may be developed from stem cells, such as, but not limited to, embryonic, induced pluripotent derived stem cells (iPSC), or organ-specific adult stem cells (ASC). Organoids derived from ASCs are generated without genetic transduction by transcription factors, unlike organoids derived from iPSCs, thus providing a more physiologically relevant in vitro model than iPSC-derived organoids. ASC-derived organoids are a functional model that can be differentiated to replicate the in vivo adult environment and can be safely transplanted into animals and humans.

Once the stem cells are isolated, they may then be grown in an extracellular matrix using a media appropriate to allow for the desired differentiation. The extracellular matrix may be a natural or synthetic extracellular matrix.

Examples of natural extracellular matrices include, but are not limited to, solubilized basement membrane preparations from Engelbreth-Hold-Swarm mouse sarcoma (MATRIGEL®), collagen, fibrin, or vitronectin.

Synthetic extracellular membranes are generally hydrogels composed of crossed linked polyethylene glycol (PEG) (for example see Nguyen et al., 2017, Versatile synthetic alternatives to MATRIGEL® for vascular toxicity screening and stem cell expansion, Nat Biomed Eng., 1: doi:10.1038/s41551-017-0096, herein incorporated by reference in its entirety). Hydrogel based extracellular matrices may provide benefits over naturally occurring extracellular matrices because the formation may be better controlled, leading to lowered lot to lot variability in desired properties.

In an embodiment, canine organoids are derived from adult epithelial stem cells. In a preferred embodiment, the stem cells are derived from the small (enteroids) and large (colonoids) intestine are produced. In a more preferred embodiment, the enteroids are produced from the ileum or jejunum. In another embodiment, the organoids are derived from urothelial cells. In some embodiments, the organoids are derived from healthy tissues. In other embodiments, the organoids are derived from diseased tissues, such as but not limited to cancer or inflammatory bowel disease.

The organoids may be produced from a human or an animal. More preferably, the organoids are produced from a carnivore, and more preferably from a canine. In a more preferred embodiment, the organoids are derived canine epithelial cells.

In some embodiment, the organoids are produced from epithelial tissue making the lining of the digestive, excretory, reproductive, or respiratory tracts. To produce the 3D cultures of canine enteroids and colonoids, leucine-rich repeat-containing G protein-coupled receptor 5 (Lgr5)-positive stem cells located in intestinal crypts may be collected from intestinal segments including the duodenum, jejunum, ileum, and colon. Additionally, in some embodiments, the cells collected may be intestinal tumors.

The epithelial organoids of the present disclosure may be cultured from various sized samples of tissue. By way of nonlimiting example, for the intestinal organoids, large whole intestinal tissue sections or from much smaller intestinal endoscopic biopsy samples from a subject using a relatively non-invasive procedure. The large whole intestinal tissue sections may be from about 1 cm to about 20 cm, from about 2.5 cm to about 15 cm, or from about 5 cm to about 10 cm. The smaller samples may be 1 mm or less, 2 mm or less, or 3 mm or less in size. The collection of the intestinal tissue may be collected in any way known in the art. For example, the tissue may be collected from living or euthanized subjects. Similar samples may be taken from other organs, such as but not limited to the stomach, lungs, or bladder.

For whole intestinal tissue sections, the tissue may then be immediately placed into a wash medium, such as, but not limited to, phosphate buffered saline (PBS) with about 1 mM to about 3 mM N-acetylcysteine, and vigorously shaken from about 3 to about 20 times, from about 5 to about 15 times, or from about 10 to about 15 times. The wash may be repeated about 3 times, about 4 times, or about 5 times or more to remove excess mucus, residual luminal contents, and other debris. After washing, the cleaned tissues may be transferred to an appropriate culture media without growth factors. While any appropriate media may be used, in a preferred embodiment, the media is complete media without growth factors (abbreviated as CMGF−) as described in the Organoid Media section. and incubated on ice.

Alternatively, the mucosal layer of the tissue samples may then be collected from intestinal tissue biopsy by any means known in the art. By way of non-limiting example, GI endoscopy biopsy forceps (Olympus America) may be used to collect the mucosa tissue samples from the whole tissue segment. This may allow up to about 15 duodenal, ileal, and colonic endoscopic biopsies to be obtained by forceps from healthy or diseased canine subjects under general anesthesia. Collected biopsies may be placed in complete media, such as, but not limited to, CMGF-medium, on ice and subjected to mechanical cleansing as described above.

Epithelial crypts containing adult intestinal stem cells may be isolated and enriched from healthy or diseased intestinal tissue. Both whole tissue samples and endoscopic biopsies are typically cut into small pieces, from about 0.5 mm to about 5 mm, from about 1 mm to about 3 mm, or from about 1 to about 2 mm in thickness with a scalpel and washed at least about 5 times, at least about 6 times, or at least about 10 times using a chelating solution. In a preferred embodiment, the chelating solution is a complete chelating solution (1×CCS) comprising from about 0.4 to about 0.6 g, from about 0.45 to about 0.55 g, or from about 0.48 to about 0.52 g Na₂HPO₄-2H₂O, from about 0.45 to about 0.65 g, from about 0.50 to about 0.6 g, or from about 0.5 to about 0.55 g KH₂PO₄, from about 2.3 to about 3.4 g, from about 2.5 to about 3.2 g, or from about 2.7 to about 3 g NaCl, from about 0.05 to about 0.75 g, from about 0.055 to about 0.07 g, or from about 0.58 to about 0.65 g KCl, from about 6.25 to about 9 g, from about 6.5 to about 8.5 g, or from about 7 to about 8 g Sucrose, and from about 4 to about 6 g, from about 4.5 to about 5.5 g, or from about 4.75 to about 5.25 g D-Sorbitol in about 500 mL water and supplemented from about 40 to about 60 μM, from about 45 to about 55 μM, or from about 50 to 55 μM DTT. While one skilled in the art will appreciate that salt solutions may be stored in concentrated form and then diluted, in a preferred embodiment, the 1× completely chelating solution may consist of a 1:5 diluted 5×CCS diluted in culture grade water, such as Milli-Q H₂O water. To prevent adherence of the cells and allow for a higher yield of cells, plastic and glass ware may be pre-wetted with 1% bovine serum albumin (BSA) throughout the procedure.

To produce the 3D cultures of canine urothelial models, cells may be collected from free-catch urine. The sample may then be centrifuged to separate out the cells of interests. Optionally, a cell sorter may be used to select certain populations of cells. This process may be used to produce other 3D cultures of epithelial cells found in suspension.

Samples of either biopsied primary tissue or from free-catch urine may then be incubated with 1×CCS containing from about 10 to about 50 mM, from about 15 to about 40 mM, or from about 20 to about 30 mM of a chelator, such as, but not limited to, methyl glycine diacetic acid (MGDA), glutamic acid N,N-diacetic acid (N.N-dicarboxymethyl glutamic acid tetrasodium salt, GLDA), nitrilotriacetic acid (NTA), diethylene triamine pentaacetic acid (DTPA), ethylenediaminetetraacetic acid (EDTA), Ethylenediamine-N,N′-disuccinic acid (EDDS), N-(1,2-dicarboxyethyl)-D,L-aspartic acid (IDS) and N-(2-hydroxyethyl)iminodiacetic acid (EDG), and salts thereof, for about 30 to about 90 minutes, for about 40 to about 80 minutes, or for about 45 to about 75 min at 4° C. on 20, 24 rpm mixer/rocker (Fisher). In a preferred embodiment, the chelator is EDTA.

After chelation, release of the epithelium cells may be augmented by trituration and/or mild vortexing in cell culture supernatant (CCS). Additional trituration and/or mild vortexing may be carried out after with the addition of fetal bovine serum (FBS; Atlanta Biologicals) to maximize release. Large fragments, such as tissue fragments, may then be allowed settled to the bottom of the tube, and the supernatant, containing the cells of interest, may then be transferred to a new conical tube and sufficiently centrifuged, for example at about 100 g, at about 125 g, at about 150 g, or about 175 g at 4° C. for about 3 minutes, for about 4 minutes, or for about 5 minutes. The pellet may then be washed with about 5 mL, about 7.5 mL, or about 10 mL complete medium, preferably CMGF−, and then sufficiently centrifuged, such as at about 60 g, at about 70 g, or about 80 g at 4° C. for about 3 minutes, for about 4 minutes, or for about 5 minutes. The pellet is then resuspended in 2 mL complete medium, and the approximate number of cells of interest isolated may be calculated using a hemocytometer.

In some embodiments, the organoids are then genetically-modified using any known technique in the art. Examples of genetic modification include DNA modification, such as but not limited to non-homologous end joining (NHEJ), homologous repair (HR) with or without the mediation of a nuclease, such as, but not limited to, Cas variants, TALEN, meganucleases, or Zinc Fingers; or RNA modifications, such as, but not limited to, RNAi, LEAPER, or Cas mediated. PCR methods, such as site directed mutagenesis may also be used for the stem cells. Transient or stable transfection with an interfering RNA may also be used to alter RNA expression in the organoids. In some embodiments, the genetic modification may be used to increase or decrease the expression of a desired protein, such as P-gp for testing drug transfer or a transporter for testing uptake of different environmental factors, or the genetic modification may alter the function of a desired protein, for example, so that P-gp or a transporter becomes resistant or susceptible to its substrate, a novel substrate, or a drug, for example, by changing the pocket size or binding sites.

The epithelial cells may then be seeded into a well comprising an appropriate extracellular matrix. In a preferred embodiment, from about 20 to about 200, from about 30 to about 150, or from about 50 to about 100 cells may be seeded in each well of a 24-well plate, wherein each well comprising about 20 μL, about 30 μL, or about 40 μL of extracellular matrix and incubated at 37° C. for about 10 minutes. However, one skilled in the art will appreciate any sized culture system may be used.

The epithelial cells may then be differentiated in the wells by adding a differentiation media. A preferred embodiment of a differentiation media comprises a complete medium with growth factors (abbreviated as CMGF+) as taught in the Organoid Media section. In a further embodiment, inhibitors may be added to the culture, forming a protective media as described in more detail in the Organoid Media section, and the organoids are incubated at 37° C. For carnivores lacking Paneth cells, the protective medium with rho kinase (ROCK) and various glycogen synthase kinase 3 (GSK-3), such as GSK3β, inhibitors may be used from about 1 days to about 4 days of intestinal stem cell culture and may enhance intestinal stem cell survival and prevent apoptosis. In a preferred embodiment, CHIR99021, an inhibitor of GSK-3, in combination with Y-27632, an inhibitor of ROCK. The inhibitors may only be added temporarily to the media for the first 2 days after isolation of intestinal crypts for enteroid/colonoid to culture and then removed. The short-term addition of the GSK-3 inhibitor, preferably CHIR99021, may enhance the initial survival and facilitated long-term propagation of enteroids/colonoids, while including Wnt3a in the media long-term improved colony forming efficiency and is required for epithelial organoid survival beyond about three passages. Removal of the ROCK and GSK-3 inhibitors from the media after the first 2 days of culture may improve differentiation of the canine epithelial organoids, such as enteroids/colonoids.

Without being bound by a specific theory, it is believed that one function of Paneth cells is to aid in differentiation. In animal species with Paneth cells, the inhibitors may be omitted.

The differentiation media, preferably CMGF+ medium, may be replenished as needed, for example every 2 days. One skilled in the art will appreciate that the changing of color of the basal media, if it contains phenol red, will signal the time to change the media. Culture may be maintained until the epithelial organoids are completely differentiated. This differentiation will vary between healthy and diseased samples, for example cancerous organoids may be solid while healthy colonoids may show a luminal compartment, crypt epithelium, and villus-like structures along with exfoliation of denuded epithelia into the lumen. To maintain continuous culture of the organoids, passage expansion may be carried out just prior to epithelial shedding as described elsewhere herein

### Organoid Media

The organoids may be grown in any acceptable media. In an embodiment, the cells may be grown in a basal media, such as but not limited to DMEM, GIBCO™ ADVANCED™ DMEM, MEM, RPMI 1640, Opti-MEM, McCoy's 5A, Hybri-Care, Leibovitz's L-15, or IMEM. The basal media may further be supplemented with nutrient mixes, such as, but not limited to F-12 and/or F-10, L-glutamine, fetal bovine serum (FBS), growth factors, additional salts, pathway inhibitors, antimicrobials, additional buffers, and/or other additives, and/or mixtures thereof to make a more complete media. Antimicrobials may include any cell culture grade antibiotics and/or antifungals. In a preferable embodiment, the media is a complete media and comprises of the basal media DMEM and is supplemented with F-12, L-glutamine, HEPES buffer, and PRIMOCIN™, available from InvivoGen (Complete Media without Growth Factors, CMGF-media), even more preferably, DMEM/F-12 supplemented with about 1 mM to about 2 mM GlutaMax-1 as an L-glutamine source, from about 5 mM to about 15 mM HEPES, and from about 80 μg/mL to 100 μg/mL PRIMOCIN™. The supplements may be added to the basal media prior to contact with the organoids or the supplements may be added after the organoids are in culture.

In another embodiment, the media is a differentiation media. In a preferred embodiment, the differentiation media includes a complete media supplemented with growth factors and/or other supplements. In a particularly preferred embodiment for epithelial organoids, the growth factors and supplements include B27 (available from Thermo Fisher Scientific), N2 (available from Thermo Fisher Scientific), epidermal growth factor (EGF), Noggin, R-spondin-1, wingless-type MMTV integration site family member 3A (Wnt3a), Gastrin, Nicotinamide, a transforming growth factor beta receptor I inhibitor (TGFβ type I), a mitogen activated protein kinase 14 (P38) inhibitor, and FBS. In a more preferred embodiment, the differentiation media is Complete Media with Growth Factors (CMGF+) supplemented with 1×B27 (Fisher), 1×N2 (Fisher), from about 0.8 mM to about 1.2 mM N-acetylcysteine, from about 40 ng/ml to about 60 ng/mL EGF, from about 80 ng/mL to about 120 ng/mL Noggin, from about 400 ng/mL to about 600 ng/mL R-spondin-1, from about 80 ng/mL to about 120 ng/mL Wnt3a, from about 8 nM to about 12 nM Gastrin, from about 8 mM to about 12 mM Nicotinamide, from about 4 mM to about 6 mM A83-01 (TGFβ type I receptor inhibitor), from about 40 μM to about 60 μM SB202190 (P38 inhibitor), and from about 6% to about 10% FBS. Without being bound by a particular theory, it is believed the inclusion of Wnt3a in the media long-term may improve colony forming efficiency and may be required for organoid survival beyond three passages. The media may include or be substantially free or free from other, known growth factors, such as but not limited to angiopoietin (ANG), bone morphogenic proteins (BMP), colony-stimulating factor (CSF), erythropoietin (EPO), fibroblast growth factor (FGF), insulin, migration-stimulating factor (MSF), myostatin (GDF-8), neuregulins, neurotrophins, interleukins, and/or placental growth factor (PGF).

In a different embodiment, the media is a protecting media. In a preferred embodiment, the protecting media is a complete media with the addition of a rho kinase (ROCK) and/or glycogen synthase kinase 3 (GSK-3) inhibitor. Rho kinase inhibitors include, but are not limited to Y27632, Y39983, Wf-536, SLx-2119, Azabenzimidazole-aminofurazans, DE-104, olefins, isoquinolines, indazoles, pyridinealkene derivatives, H-1152P, ROKα inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, and fasudil. Many GSK-3 inhibitors are known in the art, the GSK-3 inhibitor is preferably an aminopyrimidine, and more preferably CHIR99021. In a preferred embodiment, the protecting media includes ROCK and GSK-3 inhibitors in CMGF+. In a more preferred embodiment, the protecting media includes from about 8 μM to about 12 μM ROCKi and from about 1.5 μM to about 3.5 μM CHIR99021. Without being bound to a particular theory, it is believed that the addition of the inhibitors may enhance the initial survival and facilitate long-term propagation of endothelial organoids if included in the initial culture. It is believed that the inhibitors take the place of Paneth cells in canines for early differentiation of the stem cells into organoids.

In yet another embodiment, the media is a “freezing media”. For example, commercial media like Recovery™ cell freezing media may be used, It has been surprisingly found that when the cells are frozen in a media comprising from about 40% to about 60% v/v CMGF+, from about 30% to about 50% v/v FBS, and from about 5% to about 15% v/v dimethyl sulfoxide (DMSO) not only the amount of time needed for cells to grow is decreased, but more are recovered when compared to commercial media.

### Two-Dimensional Membrane Models

In an embodiment, after the organoids have formed, they may be further used to make two dimensional (2D) membrane models. This may allow easier access to the lumen or to expose each side of the organoid to a different media or environments. The organoids are first lysed into a single cell mix. Lysis may be achieved by either mechanically or chemically disrupting the organoids, such as mechanical pipetting or using trypsin. The single cell mix is then diluted to a concentration of about 1×10³ cells/mL 1×10⁴ cells/mL, about 1×10⁵ cells/mL, about 1×10⁶ cells/mL, or about 1×10⁷ cells/mL. An appropriate number of cells are then transferred onto a membrane, preferably a permeable membrane, or into a well of a TRANSWELL® plate. The cells may be transferred and cultured in an appropriate extracellular matrix for about 1 hour, for about 2 hours, or for about 3 hours. The cells are then washed and cultured for about 8 to about 16 days, from about 10 to about 14 days, or from about 12 to 13 days.

In one embodiment, a method of making a P-gp model further comprises lysing an intestinal organoid into single cells; transferring into a TRANSWELL® well; and culturing.

In an embodiment, the membranes are permeable. In a further embodiment, the membrane may be part of a microfluidics system. In an embodiment, the microfluidics system has a single chamber for the introduction of media to one side of the membrane. In another embodiment, the microfluidics system has two chambers for media on either side of the membrane allowing media to be introduced to both sides of the membrane. In an embodiment, the two chambers are filled with the same media. In another embodiment, each chamber is filled with different media.

### Methods for Passaging, Freezing, and Recovering Organoids

While any acceptable passaging, freezing, or recovery protocol may be used for the organoids, it has been surprising found that certain methods and compositions increase cell yield and growth efficacy of the organoids. The methods presented are for 24 well culture plates. One skilled in the art will appreciate that the volumes and cell densities involved will change depending on the size of the culture plate being used and can scale up or down as necessary.

### Organoid Passage

Usually after about 4 to about 7 days, the organoids are ready to be passaged. An exemplary method of passaging and cleaning the cells for a single well of a 24 well culture plate comprises:
1. Remove medium from wells (around the solid extracellular matrix) with, for example, a P1000 pipet, 5 ml pipet, or Pasteur pipet/aspirator vacuum.
2. Add about 300 μl to about 800 μl, from about 400 μl to about 700 from about 450 μl to about 550 μl cold complete media, such as, but not limited to, CMGF- or DMEM/F12, to the well and mechanically break up the extracellular matrix with pipetting, preferably with a large pipette, such as a P1000 or P5000, by pipetting up and down a sufficient number of times, for example 3 or 4 times.
3. Transfer organoids and media to a centrifuge tube, preferably a 15 ml conical tube.
4. Spin down in refrigerated centrifuge sufficiently to pellet the cells, for example at about 100 g for about 5 min at about 4° C.
5. Remove the supernatant and resuspend pellet in about 0.7 ml to about 1.5 ml protease solution, preferably trypsin or TrypLE Express, and put tube in 37° C. water bath for about 7 to about 10 minutes.
6. Add about 4 ml to about 5 ml complete media, by way of nonlimiting example DMEM/F12 or CMGF−, to stop dissociation of cells.
7. Spin down in a centrifuge to pellet the cells, for example at 100 g for 5 min at 4° C.
8. Remove the supernatant though aspiration, for example by using a 5 ml or 10 ml pipet, then P1000 or P200 pipet or an aspirator to pull the media off the pellet. Keep tube on ice.
9. Resuspend organoid pellet in an extracellular matrix (calculate the amount of extracellular matrix you will need, preferably about 25 μl/well to about 30 μl/well) using cold pipet.
10. Pipet designated amount of μl/well, for a well on a 24 well plate, it is preferable to use from about 25 μl to about 30 μl/well) of organoid/extracellular matrix mixture as a droplet using a P20, P100, or P200 cold pipet tip. Transfer plate/dish into 37° C. 5% CO₂ incubator. Let matrix settle for about 5 to about 20 minutes, add about 300 μl to about 800 μl of room temperature differentiation media, preferably CMGF+, to each well and culture in 37° C. incubator. Optionally, may use a conditioned differentiation media that is about 40% to about 60% conditioned medium (CM from WRN cells) and about 40% to about 60% differentiation media, may need to sterile filter media. Can either put in same number of wells or split 1:2 to 1:4, depending on organoid density.
11. Refresh culture with differentiation media as needed, preferably every other day.

### Clean Up Organoids

After about 2 to about 4 days, organoids passaged with a protease, such as trypsin or TrypLE, may need to be cleaned up to remove debris, dead cells, and single cells (usually differentiated cells). To clean the cells, follow the steps to passage the organoids as above, omitting steps #6-8. Can either put in same number of wells or split 1:2 to 1:4, depending on organoid density.

### Organoid Freezing Protocol

Any freezing media may be used to freeze the cells using methods known in art. However, it has been surprisingly found that the freezing media described in the Organoid Media section increases cell viability. If using a 24 well culture plate, it is preferable to increase the cell concentration in a cryovial by coming two or more wells. Usually after about 2 or 3 days after passaging as described above (unless they need clean-up to remove debris), organoids may be frozen. A preferable, exemplary method for 24 well plates of freezing cells to improve recovery comprises:
1. Remove medium from wells (around the solid extracellular matrix) using, for example, a P1000 pipet, 5 ml pipet or Pasteur pipet/vacuum aspirator.
2. Add about 300 μl to about 800 μl cold complete media, preferably CMGF- or DMEM/F12, to well and mechanically break up the extracellular matrix, preferably by pipetting up and down a sufficient number of times
3. Spin down in refrigerated swing rotor centrifuge to pellet the cells, for example at 100 g for 5 min at 4° C.
4. Remove all medium. Keep tube on ice.
5. Resuspend enteroids into freezing medium (using about 300 μl to about 800 μl for each cryovial) at original ratio of 2 wells into 1 vial, if using a 24 well plate.
6. As an optional step, before placing into liquid nitrogen, the cryovial may be kept at below about −76° C. in a freezer, preferably in a −80° C. freezer, for up to 1 week. Then transfer vials into liquid nitrogen for long-term storage.

### Organoid Revival Protocol

Any method may be used to revive (thaw) organoids from liquid nitrogen. However, it has been surprisingly found that the number of cells recovered and the amount of time it takes to grow the organoids may be improved by:
1. Thaw an extracellular matrix aliquot on ice in black anodized aluminum cooling block and pre-warm plate
2. Add about 8 ml to about 10 ml of a complete media, such as, but not limited to, CMGF- or DMEM/F12, into a 15 ml tube, leave tube on ice
3. Optionally, transfer frozen vial containing organoids from liquid nitrogen to dry ice
4. Swirl vial in about 37° C. water until thawed (liquid)
5. Immediately transfer contents in the vial to 15 ml tube containing about 10 ml cold complete media drop by drop
6. Spin down a centrifuge at 100 g for 5 min at 4° C.
7. Remove medium and leave 15 ml tube containing organoid pellet on ice. Resuspend pellet in about 60 μl to about 120 μl of extracellular matrix (enough to seed about 4 wells with 15 μl to about 30 μl/well of matrix) using a cold pipet tip, plate organoids as droplets in 4 wells of 24 well plate and transfer plate to 37° C. incubator.
8. Let gel settle for 5-20 minutes, add about 300 μl to about 800 μl of room temperature protecting media to each well and culture in 37° C. incubator.
9. Refresh culture with differentiation media as needed, preferably every other day, until ready to be passaged (typically 5-7 days).

### Models and Methods of Use

### Methods for Use in Diseases

The organoid models described above may be used in detecting differences in organoids due to disease by detecting changes in RNA or protein expression or detecting changes in concentrations of metabolites within the organoids or within the culture media. For example, tissue samples may be taken from a diseased subject and differences in RNA or protein production may be detected in comparison to a control subject lacking the disease. Alternatively, both the diseased and control samples may be derived from the same subject to detect within subject differences. Detecting difference from the within subject comparison may show how the disease developed locally more clearly than an across subject comparison.

Alternatively, genetically-modified organoids may be used to determine the role of genes which may be the cause of the disease or which may provide resistance to a disease. For example, if a knockout of a protein, such as, but not limited to, a transcription factor or DNA repair gene results in immortalization or tumor development in a healthy sample, it may be concluded that that protein is a proto-oncogene in the epithelial tissue.

In an embodiment, the organoids and methods of use provide an effective model for identifying differences from human models and animal models, preferably for canine species. This is particularly important when differences between humans and canines emerge. The organoids and methods of use provide an ex vivo model for use in canine species. The three-dimensional culture conditions provide effective tools for modeling healthy and diseased subject response to an environmental or dietary trigger.

In an embodiment, to determine if there is an age difference in the response to a trigger, serial samples of stem cells may be taken from the same subject to produce longitudinal studies. Some carnivores, such as canines, due to their shorter lifespan but similar habitual diets compared to humans, may beneficially provide a more rapid development of a model for chronic diseases which may how an environmental or dietary trigger interacts in vivo over time.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Methods for Use in Environment Responses

The organoid models described above may be used in detecting changes to the organoids due to environmental changes by detecting changes in RNA or protein expression, changes in epigenetics, such as DNA methylation or histone modifications, or detecting changes in concentrations of environmental factors or their metabolites. For example, an environmental or dietary trigger may be added to the media and the epithelial organoids may be used to measure the transport and metabolism of the trigger from surrounding media to estimate the apparent permeability and intestinal metabolism of the trigger. The trigger may be any environmental or dietary trigger, such as, but not limited to, pathogens or their components, such as whole bacteria, viruses, or paramecium or components such as lipopolysaccharide or viral proteins; heavy metals; chemicals, such as volatile organic compounds, phthalates, or formaldehyde; or small molecules, such as carbon monoxide, arsenic, or cyanide.

Alternatively, genetically-modified organoids may be used to determine the role of genes which may be responsible for the uptake or metabolism of environmental or dietary trigger. For example, if a knockout of a transporter protein reduces the removal of the trigger from solution while overexpression increases removal, then it may be concluded that that transporter may at least partially transport the trigger, depending on the change in removal.

The organoids may be used by measuring the rate or amount of trigger may be taken into the interior of the organoid. Similarly, an organoid cultured on a permeable membrane, such as a TRANSWELL® plate, may be used to measure transfer of the trigger across the membrane.

In other embodiments, a trigger may be added to the culture media and then the media sampled to detect changes in compounds known to be produced by the organoids. This detection may show what downstream effect the trigger has on the epithelial tissue from with the organoid derives.

In an embodiment, the organoids and methods of use provide an effective model for identifying differences from human models and animal models, preferably for canine species. This is particularly important when differences between humans and canines emerge. The organoids and methods of use provide an ex vivo model for use in canine species. The three-dimensional culture conditions provide effective tools for modeling healthy and diseased subject response to a trigger.

In an embodiment, to determine if there is an age difference in the response to a trigger, serial samples of stem cells may be taken from the same subject to produce longitudinal studies. Canine, due to their shorter lifespan but similar diets compared to humans, may beneficially provide a more rapid development of a model for chronic diseases which may be used to investigate how a trigger interacts in vivo over time.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Methods for Use in Diet and Diet Changes

The organoid models described above may be used in detecting changes to the organoids due to changes in diet or additives to a diet by detecting changes in RNA or protein expression, changes in epigenetics, such as DNA methylation or histone modifications, or detecting changes in concentrations of metabolites. For example, an initial diet may be provided to the organoid followed by removal of a compound or the addition of a compound. Change in gene or protein expression or the concentrations of metabolites within the cells or media may then be detected. A detected change may allow for measuring the effects a change in diet has on energy levels or toxicity of a given diet or additive.

Alternatively, genetically-modified organoids may be used to determine the role of genes which may be responsible for the uptake or metabolism of dietary compounds. For example, if a knockout of a transporter protein reduces the removal of a compound found within the diet from solution while overexpression increases removal, then it may be concluded that that transporter may at least partially transport the dietary compound, depending on the change in removal.

The organoids may be used by measuring the rate or amount of the diet or a component thereof may be taken into the interior of the organoid. Similarly, an organoid cultured on a permeable membrane, such as a TRANSWELL® plate, may be used to measure transfer of the dietary compound across the membrane.

In an embodiment, the organoids and methods of use provide an effective model for identifying differences from human models and animal models, preferably for canine species. This is particularly important when differences between humans and canines emerge. The organoids and methods of use provide an ex vivo model for use in canine species. The three-dimensional culture conditions provide effective tools for modeling healthy and diseased subject response to a diet or a change in diet.

In an embodiment, to determine if there is an age difference in the response or ability to metabolize to a given diet or a compound within the diet, serial samples of stem cells may be taken from the same subject to produce longitudinal studies. Canine, due to their shorter lifespan but similar diets compared to humans, may beneficially provide a more rapid development of a model for how a subject is capable of metabolizing a diet over time.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Models for P-Glycoprotein-Mediated Drug Transport

The above organoids may be used to make diverse models, which can be used for assaying P-gp mediated drug transport.

In some embodiments, the model for P-gp transport comprise intestinal organoids, wherein the organoids are differentiated from Lgr5-positive stem cells. In preferred embodiments, the Lgr5-positive stem cells are obtained from canines.

In some embodiments the organoids express wild-type levels P-gp. In other embodiments the organoids have been genetically-modified to alter the expression of P-gp. In some embodiments, the genetic modification knockdowns, knockouts, or overexpresses P-gp.

In further embodiments, the organoids are cultured in a monolayer on a TRANSWELL® membrane. In an embodiment, the TRANSWELL® membrane is permeable.

In some embodiments, the models include a P-gp inhibitor. P-gp inhibitors include, but are not limited to, amiodarone, clarithromycin, ciclosporin, colchicine, diltiazem, erythromycin, felodipine, ketoconazole, lansoprazole, omeprazole and other proton-pump inhibitors, nifedipine, paroxetine, reserpine, saquinavir, sertraline, quinidine, tamoxifen, verapamil, duloxetine, elacridar, CP 100356, zosuquidar, tariquidar, valspodar and reversan.

In other embodiments, the models include a P-gp inducer. P-gp inducers include, but are not limited to, carbamazepine, dexamethasone, doxorubicin, nefazodone, phenobarbital, phenytoin, prazosin, rifampicin, St. John's wort, tenofovir, tipranavir, trazodone, and vinblastine.

In yet other embodiments, the models include a P-gp substrate. Substrates of P-gp are susceptible to changes in pharmacokinetics due to drug interactions with P-gp inhibitors or inducers. Some of these substrates include colchicine, ciclosporin, dabigatran, digoxin, diltiazem, fexofenadine, indinavir, morphine, and sirolimus.

### Methods of Making P-Glycoprotein Models

Traditional 2D cell cultures involving immortalized cells, such as cancer cells with or without a genetic modification to express specific proteins, such as, but not limited to, P-gp, or primary cells have been used in coverslip or standard wells. However, the absence of a basolateral compartment precludes cell polarization and may prevent the study of transport across cell layers. Further, the use of cancer cells, or other immortalized cells, or genetic modification may lead to changes in expression of protein when compared to the normal physiological state. Therefore, 3D models using cells differentiated from initial stem cells may result in models which are more like the normal physiological state than 2D models. Such models include, but are not limited to, 3D organoids and TRANSWELL® cultures.

In an embodiment, the organoid models may be used to study P-gp and drug permeability, efficacy, and safety. For P-gp models, a sample of organoids may be taken and the expression and/or localization of P-gp nucleic acid or protein may be assayed. In some embodiments, PCR may be used to detect the expression of P-gp RNA. In other embodiments, immunohistochemistry (IHC) may be used to measure the expression and/or localization of P-gp protein. If the organoids have been genetically-modified, then quantitative PCR or IHC/immunofluorescence may be used to quantify the change in expression of P-gp.

In an embodiment, a method of making a P-gp model comprises: obtaining an intestinal sample; extracting leucine-rich repeat-containing G protein-coupled receptor 5 (Lgr5)-positive stem cells located in intestinal crypts; culturing said Lgr5-positive stem cells within an extracellular matrix, wherein the culture media causes differentiation of the stem cells; maintaining the culture until organoids form, wherein the organoids are positive for P-gp expression.

In further embodiments, the stem cells are genetically-modified. In some embodiments the genetic modification knockdowns the expression of P-gp. In other embodiments the genetic modification overexpressed P-gp. In yet other embodiments the genetic modification alters the cellular location of P-gp. In yet another embodiment, the genetic modification mimics mutation in a disease.

In a preferred embodiment, the intestinal sample is obtained from a canine subject.

In an embodiment, the intestinal sample is from the ileum. In another embodiment, the intestinal sample is from the jejunum. In yet another embodiment, the intestinal sample is from the colon.

### Use of Models in Drug Development and Screening

The above compositions may be used in drug development and screening by measuring transport (i.e. drug efflux) through transporters, such as, but not limited to, P-gp. For example, the intestinal organoids may be used to measure the intestinal transport and metabolism of a compound from surrounding media to estimate the apparent permeability and intestinal metabolism of the compound. The compound may be a drug or a P-gp substrate. If the compound is a test drug, then a P-gp inhibitor or inducer may be co-administered with the drug to determine if P-gp transports the drug out of solution by measuring an increase or decrease in drug permeability, respectively. Since P-gp is an efflux protein, inhibiting P-gp-mediated drug transport will result in an increase in drug permeability from the donor i.e. apical to the receiver i.e. basal side of the TRANSWELL®. Alternatively, genetically-modified organoids may be used to determine the role of P-gp on said drugs removal. For example, if a knockout reduces the removal from solution while overexpression increases removal, then it may be concluded that P-gp may at least partially transport the drug, depending on the change in removal.

If the compound is a P-gp substrate, then a drug may be co-administered with the substrate in order to determine which of the drugs may interfere with P-gp mediated transport of the substrate out of solution by observing a change in the rate of removal from solution.

The organoids may be used to estimate intestinal permeability by measuring the rate or amount of substrate or drug taken into the interior of the organoid. Similarly, an organoid cultured on a permeable membrane, such as a TRANSWELL® plate, may be used to measure transfer of the drug across the membrane.

In an embodiment, the organoids and methods of use can be used to assess a variety of therapeutic drugs. In an embodiment, exemplary therapeutic drugs include, nonsteroidal anti-inflammatory drugs (NSAIDs), chemotherapy drugs, etc. Any candidate drug may be tested, preferably the drug molecules from the Biopharmaceuticals Classification System (BCS). See Amidon G L, et al., 1995, A Theoretical Basis For a Biopharmaceutics Drug Classification: The Correlation of In Vitro Drug Product Dissolution and In Vivo Bioavailability, Pharm Res, 12: 413-420. The BCS is a scientific framework for classifying drug substances based on their aqueous solubility and intestinal permeability. When combined with the dissolution of the drug product, the BCS takes into account three major factors that govern the rate and extent of drug absorption from IR solid oral dosage forms: (1) dissolution, (2) solubility, and (3) intestinal permeability.

According to the BCS, drug substances are classified as follows:
1. Class 1: High Solubility-High Permeability
2. Class 2: Low Solubility-High Permeability
3. Class 3: High Solubility-Low Permeability
4. Class 4: Low Solubility-Low Permeability.

In an exemplary embodiment, the organoids and methods of use described herein can provide effective models to assess therapeutic efficacy of such exemplary therapeutic drugs including, nonsteroidal anti-inflammatory drugs (NSAIDs), chemotherapy drugs, etc. In a further embodiment the organoids and methods of use described herein can assess therapeutic failures and toxicity, including exposure-associated toxicity, of such exemplary therapeutic drugs including, nonsteroidal anti-inflammatory drugs (NSAIDs), chemotherapy drugs, etc. In still further embodiments, the organoids and methods of use described herein can assess how the exemplary therapeutic drugs will affect the intestines of a subject, providing ability to determine any rate limiting dosages of the therapeutic drugs.

In an embodiment, the organoids and methods of use provide an effective model for identifying differences from human models and animal models, namely for canine species. This is particularly important when differences between humans and canines emerge. The organoids and methods of use provide an ex vivo model for use in canine species. The three-dimensional culture conditions provide effective tools for modeling healthy and diseased subject oral absorption and/or elimination of drugs.

In an embodiment, serial samples of stem cells may be taken from the same subject to produce longitudinal studies. Canine, due to their shorter lifespan but similar diets compared to humans, may beneficially provide a more rapid development of a model for chronic diseases which may how a drug interacts in vivo over time.

In an embodiment, canine and intestinal stem cells taken from healthy dogs provide an accurate predictor of the efficacy of the therapeutic drugs being tested as they closely mimic biological responses and physiologic state in dogs, providing a good predictor of therapeutic efficacy in vivo based on cells produced in vitro. In a still further embodiment, canine and intestinal epithelial cells taken from diseased dogs better predict the efficacy of the therapeutic drugs being tested and more closely mimic biological responses and the physiological state in such diseased dogs, providing a good predictor of therapeutic efficacy in vivo based on cells produced in vitro. Such methods of screening of potential therapeutic drugs and screening of drug responses in ex vivo models beneficially speed up the drug testing timeline to trials as well as provide a better predictor of efficacy in the canines with similar diseases to the animals that the canine cells were taken from for producing the organoids.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Additional Uses

One skilled in the art will appreciate that even more complex experimental designs are possible with the organoids. For example, the interaction between diet, treatment, and disease may be determined by combining the methods relating to each design. More specifically, a nonlimiting example of a more complex design may be to detect cell viability between a healthy population of organoids receiving a specified diet, a diseased population of organoids receiving the same specified diet, a healthy population of organoids receiving a higher protein or fat diet, and a diseased population of organoids receiving the same higher protein or fat diet. Additionally, the organoids could further be treated with, for example, a chemotherapy regime if the disease is cancer. This may allow one to determine if there are any interaction effects among diet, disease, and treatment. Further considerations may also include longitudinal studies as described above to determine if age may play a role in any interaction effects.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Developing systems for studying drug intestinal transport and metabolism is critical for predicting bioavailability of therapeutic drugs in medicine. Specifically, conventional systems such as 2D epithelial cultures utilizing cancer-derived cell lines (e.g. Caco-2, T84, and HT29), or spontaneously immortalized epithelial cells (e.g. Rat Intestinal Epithelial [RIE] cultures) do not faithfully reproduce the structure and function of the intestinal epithelium. Since such systems do not express canine P-gp, there is the risk for incorrect conclusions associated with substrate specificity, drug-drug interactions, or enzyme kinetics. Specifically, Caco-2 cells are a human colon adenocarcinoma cell line and are not derived from canine tissues. Therefore, any data generated using Caco-2 will have uncertain relevance to models of canine oral drug absorption and metabolism.

Because in vitro 3D cell culture systems provide a more realistic translation to in vivo conditions than do most 2D culture systems, 3D enteroids will better harness the complexity of the in vivo biology. Accordingly, this would provide an opportunity to conduct in vitro mechanistic studies for evaluating drug absorption. However, the molecular characteristics of the enteroids has not been assessed, particularly for P-gp. Therefore, it is essential to assess the localization, expression, and function of P-gp in 3D models, such as canine ileal enteroids.

### Material and Methods

### Study Dogs

Eight healthy spayed female Beagle dogs, 1 year of age with an average body weight of 8.57±0.93 kg, were enrolled in this study. All dogs were housed at the College of Veterinary Medicine at Iowa State University in temperature-controlled rooms (20° C.) on a 12:12 hour light: dark schedule. Suitability for inclusion was evaluated by clinical examination as well as by measuring complete blood count (CBC) and chemistry panels (i.e. albumin, total protein, alanine aminotransferase, aspartate aminotransferase, blood urea nitrogen, and creatinine). All dogs were clinically healthy, and all blood parameters remained within reference intervals during the study.

### Intestinal Tissue and Stem Cell Isolation for Culture of Ileal Enteroids

Intestinal biopsies were obtained endoscopically for intestinal stem cell isolation and histological evaluation from healthy research colony dogs. All animal procedures in this study were approved by the Iowa State University Institutional Animal Care and Use Committee (IACUC protocol: 9-17-8605-K). Representative ileal biopsies (as defined anatomically during endoscopic examination) were fixed with 10% formalin and stored in 70% ethanol for immunohistochemistry (IHC) staining. Epithelial crypts containing primary adult intestinal stem cell (ISC) were isolated and cultured as previously described13. Briefly, endoscopic biopsy samples were cut into small pieces and intestinal crypt cells were released by incubating the samples with complete chelating solution and EDTA (30 mM) for 60 min at 4° C. After crypt release, the crypt-containing pellet was suspended and seeded in 30 μL per well of MATRIGEL® (CORNING® MATRIGEL® Growth Factor Reduced (GFR) Basement Membrane Matrix) and 500 μL per well of complete medium with ISC growth factors (CMGF+) supplemented with 10 μM rho-associated kinase inhibitor (ROCKi) Y-27632 (Stem-Gent) and 2.5 μM glycogen synthase kinase (GSK3β) inhibitor CHIR99021 (StemGent) before the plate was incubated at 37° C.13. The culture medium was changed to CMGF+ without any supplement after 2 days of crypt isolation, while passage and expansion of enteroids were performed with TrypLE Express treatment at 37° C. for 10 min. Once stable enteroids cultures were established, representative segments of ileal enteroids were fixed with 10% formalin and stored in 70% ethanol for IHC staining. All the formalin fixed samples were paraffin embedded and cut into 3-μm sections for placement onto glass slides.

### Immunohistochemistry (IHC)

Immunohistochemistry (IHC) assays were performed based on a commercially available protocol at the Iowa State University Veterinary Diagnostic Laboratory (Discovery Ultra, Ventana Medical Systems, Inc.). Briefly, paraffin-embedded sections were first deparaffinized and rehydrated, followed by antigen retrieval and blocking steps. The sections were incubated with primary antibodies (Anti-P-gp antibody, PAS-61300, ThermoFisher, MA) at 1:1600 dilution, followed by Diaminobenzidine (DAB) staining reagents and subsequently treated with hematoxylin counterstaining. Image acquisition was performed using the Olympus CellSens Standard Ver.1.18 (Tokyo, Japan), while semi-quantitative image analysis of DAB detection was performed using the ImageJ v1.52q15. The quantified DAB staining was controlled by the hematoxylin counterstaining to control for the number variation of the cell number within an image.

### Statistical Analysis

Shapiro-Wilk tests were used to assess the normality of the data. The Mann-Whitney U test was used to compare the positive staining level between original intestinal tissues and enteroids for each dog.

### Results

### Consistent Apical Localization of P-gp in Ileal Tissues and Ileal Enteroids

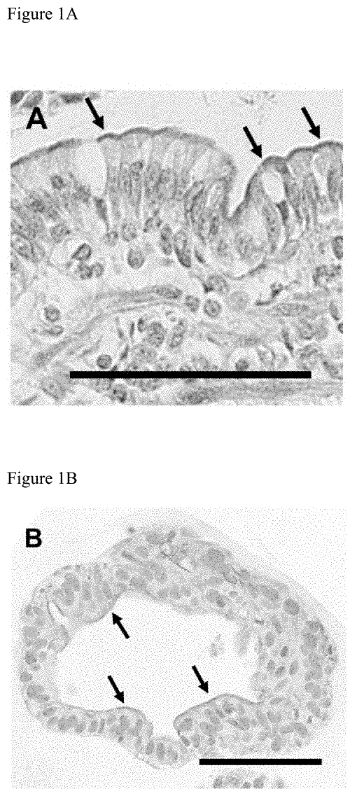

P-gp expression data in ileal tissues and ileal enteroids as evaluated by IHC are presented in FIG. 1. As the arrowheads indicate, P-gp efflux proteins were consistently expressed on the apical surface of the ileal epithelium (FIG. 1A) and of the ileal enteroids (FIG. 1B).

### Similar Expression Levels of P-gp Between Ileal Tissues and Ileal Enteroids

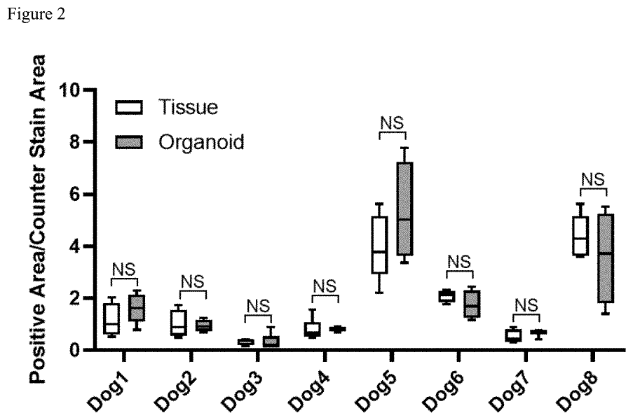

P-gp expression in ileal tissues and ileal enteroids, as evaluated by IHC, was semi-quantified using ImageJ and no statistically significant differences were found in P-gp expression between ileal enteroids and ileal tissues from the same dogs (P>0.9999) (FIG. 2). The slightly higher variability observed in the enteroids vs the intestinal tissues of some dogs may reflect individuals exhibiting a lack of homogeneity in the density of P-gp across ileal samples. It may also be a function of the difference in sample numbers (10 tissue sections, 20 ileal organoids) derived from each dog.

### Conclusion

These results show that expression of P-gp in the enteroids, while having a slightly higher variation in expression, are expressed in the same location in the same amount as tissue samples. This supports that use of these models as a potential for use in studying intestinal transport. However, it does not show that the P-gp of the enteroids maintain their function.

### Example 2

As shown above, the enteroids have the same expression of P-gp, but it is unknown if the transport ability has been maintained and was therefore tested using a substrate and an inhibitor.

### Materials and Methods

### Rhodamine 123 (Rh123) Transport Studies in Canine Real Enteroids

Canine ileal enteroids were passaged into 15 μL per well of MATRIGEL® in chambered cover glass system (Lab-Tek, Chambered Coverglass 8 well) and cultured with 300 μL per well of CMGF+ media for 2 days. Rhodamine 123 (Rh123) was used as a P-gp substrate at increasing concentrations of 1, 10, 20, and 50 μM, while verapamil was used as a P-gp inhibitor at the nominal concentration of 20 μM16. Rh123 transport experiments were divided into: (1) Control [CTR] (Rh123 alone, N≥20 enteroids/well, 10 wells total) vs. (2) Treatment [TRT] groups (Rh123 plus verapamil, N≥20 enteroids/well, 10 wells total). Enteroids were treated for 30 min and then washed with room-temperature PBS. During our preliminary study assessing P-gp transport at 60 min incubation time, the inhibitory effects of verapamil were less pronounced due to saturation of Rh123 within the luminal aspect of the enteroids (data not shown); therefore, a 30 min time point was chosen for this study.

Confocal microscopy (SP5 X MP LAS X; Leica) at the Iowa State University Roy J. Carver High Resolution Microscopy Facility was used to acquire images to detect green fluorescence within the luminal aspect of the enteroids. Fluorescence intensity was controlled by the luminal area to control for the size variation of the enteroids and quantified using the ImageJ software.

### Statistical Analysis

An ANOVA with Turkey's multiple comparison test was used to compare the means of the CTR group for increasing doses of Rh123 in absence of verapamil. A linear regression analysis was used to compare study groups (CTR vs. TRT) to assess the overall effect of verapamil. Student's t-tests were used to compare study groups (CTR vs. TRT) for the various doses of Rh123. The Benjamini-Hochberg Procedure was performed to control the risk of false positive for multiple comparisons. All statistical analyses were perform using GraphPad Prism 8.2.1 (San Diego, Calif.) and R 3.5.1. P<0.05 were considered as statistically significant.

### Results

### Functional Assessment of P-Gp in Ileal Enteroids

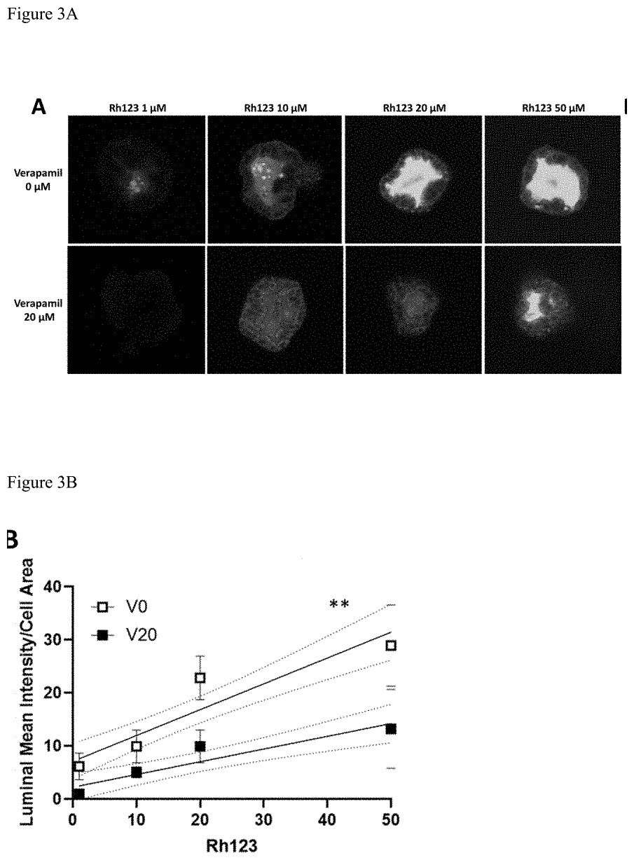

Findings on Rh123 transport inhibition with verapamil are summarized in FIG. 3. An accumulation of green fluorescent Rh123, a P-gp substrate, is typically observed within the luminal space of enteroids when the P-gp protein is functional. Conversely, the accumulation of rhodamine is inhibited if the transporter function is blocked by a P-gp inhibitor such as verapamil.

In brief, there was a significant dose response with Rh123 luminal transportation between 1 and 10 μM (P<0.0001), 1 and 50 μM (P<0.0001), 10 and 20 μM (P<0.05), and 10 and 50 μM (P<0.001) after 30 min incubation even without verapamil. Also, there was a significant inhibition throughout the different concentrations of Rh123 after 30 min incubation with 20 μM of verapamil (P<0.01) (FIG. 3B). Results from the linear regression analysis showed a significant effect of verapamil on luminal transportation (**P<0.01), as confirmed by the significant slope difference between CNT and TRT groups. In addition, significant inhibition of the luminal transport of Rh123 with 20 μM of verapamil was noted at 1 μM (P<0.01), 10 μM (P<0.01), 20 μM (P<0.01), and 50 μM (P<0.05) of Rh123. Also, significant slope difference was determined by a linear regression analysis (**P<0.01). It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. In addition, the contents of all patent publications discussed supra are incorporated in their entirety by this reference.

### Conclusion

These results demonstrated that amounts of Rh123 ranging from 1 to 20 μM of Rh123 is sufficient to allow for measuring the inhibition effect of 20 μM verapamil after 30 min incubation. Therefore, P-gp of the enteroids have maintained their transportation ability. This supports that use of these models as a potential for use in studying intestinal transport.

### Example 3

The multi-drug resistance protein gene (ABCB1, MDR1) codes for P-glycoprotein (P-gp), an important drug efflux transporter which detoxifies cells and is involved in resistance to antimicrobial and chemotherapy drugs. Inhibition of P-gp by some drugs can also lead to dangerous adverse effects as well as harmful drug-drug interactions, making characterization of P-gp-mediated drug transport of candidate drugs critical during the preclinical evaluation phase. MDR1 mutations are common in some breeds of dogs, making them vulnerable to severe side effects or death from multiple common veterinary therapeutics, including ivermectin and chemotherapeutics. In this study, we used CRISPR/CAS9 gene editing to knockout MDR1 in canine intestinal organoids as described in Example 1, as a model to mimic the commonly reported MDR1 deletion mutation in dogs.

### Methods

Characterization of P-gp function in canine enteroids was performed by incubation with 10 μM rhodamine123 (Rh123), a fluorescent dye substrate for P-gp, and/or 20 μM verapamil, a P-gp inhibitor, for 30 minutes. P-gp function was quantitated by fluorescent microscopy and ImageJ. MDR1 knockout in canine enteroids was achieved by Lipofectamine transfection with a CRISPR/Cas9 all-in-one plasmid specific for MDR1. Transfection efficiency was monitored by green fluorescent protein (GFP) expression in both control and CRISPR/Cas9 plasmid.

### Results

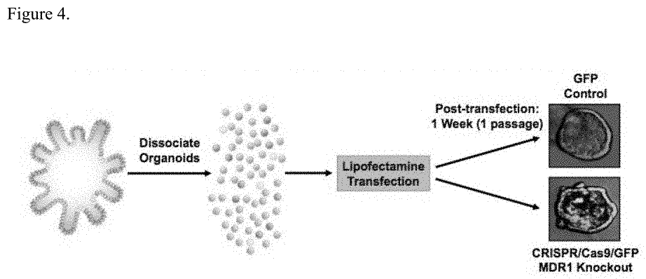

Canine 3D enteroids were stably transfected with control GFP plasmid or with a CRISPR/Cas9 plasmid to knockout MDR1. Transfection of organoids with both plasmids was maintained for multiple passages, as confirmed by fluorescent microscopy (FIG. 4).

### Conclusions

Knockout of MDR1 expression in canine intestinal organoids mimics MDR1 mutations in some dog breeds and will be a useful model for pharmaceutical drug toxicity and uptake studies. Methods developed for the stable transfection of canine intestinal organoids expand their utility for mechanistic drug screening, efficacy, toxicity, and interaction studies for many human diseases, including cystic fibrosis, inflammatory bowel disease, and colorectal cancer.

### Example 4

The 3D organoid body presents certain considerations for direct access to the lumen for studying the luminal cell interactions with dietary constituents, microorganisms, drugs, or environmental or dietary triggers transported through an epithelial layer. While microinjection of a luminal component (e.g., living bacterial cells) into the lumen of an organoid may be used with the 3D organoids, the technique can be require special considerations due to the heterogeneity in organoid size, invasive injection, and the requirement of techniques and equipment. Thus, cultures of a polarized, two-dimensional, intestinal cell monolayer may be better suited for the standardized measurement of transepithelial permeability and epithelial-luminal interaction due to easier accessibility of the apical surface. Moreover, creating a canine-derived intestinal interface may be further improved by integrating the optimized protocol to the intestinal microphysiological systems.

Therefore, a method for generating an intact monolayer of the canine colonoid-derived epithelium was developed. The characterization of the formed epithelial monolayer that provides an accessible tissue interface, polarization, lineage-dependent epithelial cell differentiation, tight junction barrier, permeability, and the expression of key efflux pump using various imaging modalities was determined.

### Materials and Methods

### Creation of a Biopsy-Derived Canine Colonoid Line

Intestinal biopsies were obtained via colonoscopy for intestinal stem cell isolation from healthy research colony dogs at the Iowa State University College of Veterinary Medicine. All animal procedures in this study were approved by the Iowa State University Institutional Animal Care and Use Committee (IACUC protocol: 9-17-8605-K). Colonic crypts containing primary adult intestinal stem cells were isolated and cultured, as previously described in Chandra et al. (Derivation of adult canine intestinal organoids for translation research in gastroenterology. BMC Biol. 2019 Apr. 11; 17(1): 33. Doi:10.1186/s12915-019-0652-6 PMID: 30975131, herein incorporated in its entirety). Briefly, endoscopic biopsy samples from colonoscopies were cut into small pieces, and intestinal crypt cells were released by incubating the samples with a complete chelating solution and EDTA (30 mM; Alfa Aesar) at 4° C. for 60 min. After the crypt release, the crypt-containing pellet was suspended and seeded in 30 μL per well of MATRIGEL® (Corning) and 500 μL per well of complete medium supplemented with intestinal stem cell (ISC) supporting factors including 10 μM rho associated kinase inhibitor (ROCKi) Y-27632 (StemGent) and 2.5 μM glycogen synthase kinase (GSK3β) inhibitor (StemGent) before the plate was incubated at 37° C. The culture medium was changed to complete medium without any supplementation after 2 days of crypt isolation.

### Colonoid Culture

A complete medium containing 10 mM HEPES (Gibco), 1×GlutaMAX (Invitrogen), 100 units/mL penicillin, and 100 μg/mL streptomycin in Advanced DMEM/F12 (Gibco) was first prepared. Conditioned medium was prepared by culturing Wnt3a-producing L cells (ATCC, CRL 2647), R-spondin1 (Rspo1) cells (Trevigen), and Noggin secreting cells (Baylor's College of Medicine), as previously described in Sato T and Clevers H (Growing Self-Organizing Mini-Guts from a Single Intestinal Stem Cell: Mechanism and Applications. Science. 2013 Jun. 7; 340(6137): 1190-4. Doi:10.1126/science.1234852 PMID: 23744940). In the complete medium, the volume ratio of basal and each conditioned medium is defined at 20/50/20/10% (v/v) and murine recombinant epidermal growth factor (EGF) (50 ng/mL; Peprotech), SB202190 (30 μM; Sigma Aldrich), A-8301 (500 nM; Sigma Aldrich), Gastrin (10 nM; Sigma Aldrich), N-acetylcysteine (1 mM; MP Biomedicals), nicotinamide (10 mM; Sigma Aldrich), N2 (1×; Gibco), and B27 (1×; Gibco) were also supplemented. The complete medium was changed every other day, and organoids were passaged once a week by mechanically breaking down the organoids, spinning down the fragmented organoids (100×g, 4° C., 5 min), resuspending centrifuged organoids with fresh MATRIGEL® on ice, and then plating them in each well of a 24 well plate (Corning).

### Culture of a Colonoid-Derived Monolayer

The 3D colonoids were harvested from MATRIGEL® after 7 days of culture by addition of EDTA solution (0.5 mM; Alfa Aesar) on ice, then transferred in 15 mL tubes and centrifuged (100×g, 4° C., 5 min). The organoid pellet was incubated in 1 mL TrypLE Express (Gibco) for 10 min while shaking at 37° C. in a water bath. The centrifuged (100×g, 4° C., 5 min) organoid fragments were resuspended in complete medium and further dissociated by repeated pipetting and subsequent filtering of the cell suspension through a cell strainer (cut-off size, 40 μm, Corning) to obtain a single-cell suspension. TRANSWELL® inserts (0.4 μm pores, Corning) were pre-coated with MATRIGEL® (100 μg/mL; Corning) and collagen I (30 μg/mL; Fisher Scientific) in PBS or basal medium at 37° C. for 1 h. Dissociated cells were counted manually using a cell counter (Hemocytometer; Hausser Scientific) and seeded at 106 cells/mL in pre-coated TRANSWELL® inserts. After 3 days of incubation in a humidified incubator at 37° C. with 5% CO2, the cell monolayer was established. The morphology of a cell monolayer was intermittently monitored for up to two weeks by phase-contrast microscopy (Axiovert 40CFL, Zeiss).

### Evaluation of the Epithelial Barrier Integrity

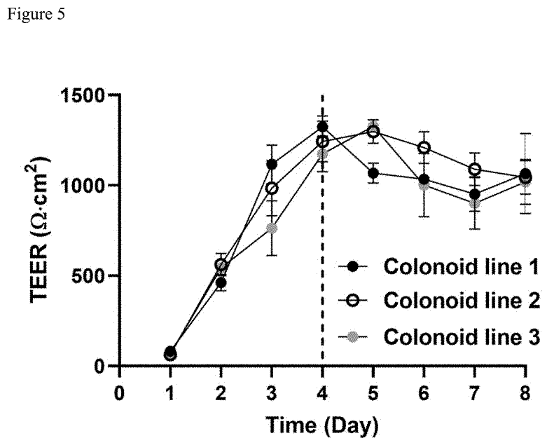

The barrier function of the intestinal epithelial monolayer was measured by monitoring TEER. The TEER value was measured by using Ag/AgCl electrodes connected to an Ohm meter (Millicell ERS-2; Millipore). Normalization of TEER was performed following the equation as, TEER=(Oₜ−O_{blank})×A, where Oₜ is the resistance (in Ohms) at the measured time point since the start of the culture; Omani, is the resistance of the blank, and A is the surface area cultured on the nanoporous insert in cm². To investigate the reproducibility in TEER values from various canine colonoid-derived monolayers, TEER measurement was performed in 2 biological replicates with 4 technical replicates using 3 different canine colonoid lines (FIG. 5). To assess the effect of culture conditions on TEER, the colonoid-derived monolayer was cultured with proliferation medium (complete medium with Wnt3a proteins) or differentiation medium (a complete medium without Wnt3a) after forming a monolayer which was at Day 4. This study was performed in 2 biological replicates with 4 technical replicates in each condition (i.e., Diff vs. Control). The medium in the TRANSWELL® insert was changed to either differentiation medium or proliferation medium while the bottom wells were filled with proliferation medium.

To assess intestinal barrier permeability, fluorescein sodium salt (Mw, 376.27 Da; 0.05 μg/mL) was used as a paracellular marker. The concentration of fluorescein that transported through the cell monolayer (from apical to basolateral) was measured by SpectraMax microplate reader (Molecular Devices). The apparent permeability (Pₐₚₚ) was calculated using the following equation: Pₐₚₚ=(dQ/dt)/(C0×A), where dQ/dt (μg/sec) is the steady-state flux, C0 (μg/mL) is the initial concentration of the fluorescein in the apical chamber, and A (cm²) is the surface area cultured on the nanoporous insert. This experiment was performed with 2 biological and 4 technical replicates.

### Immunofluorescence Imaging

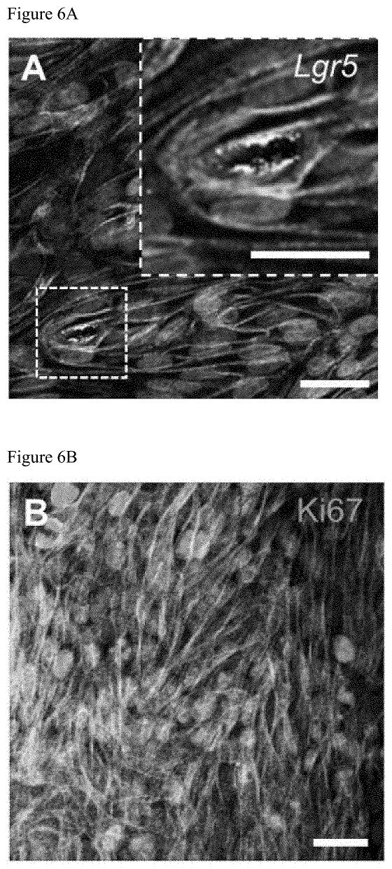

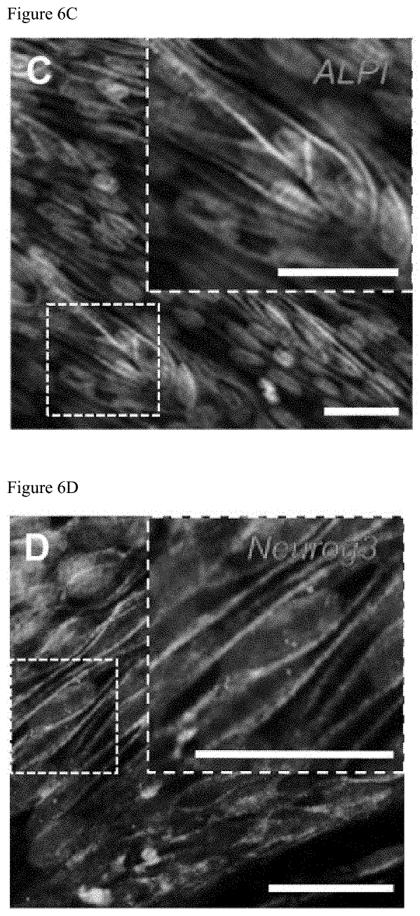

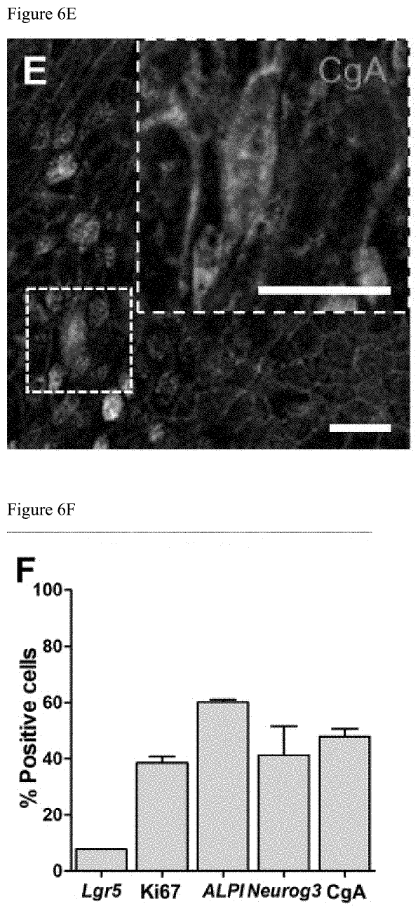

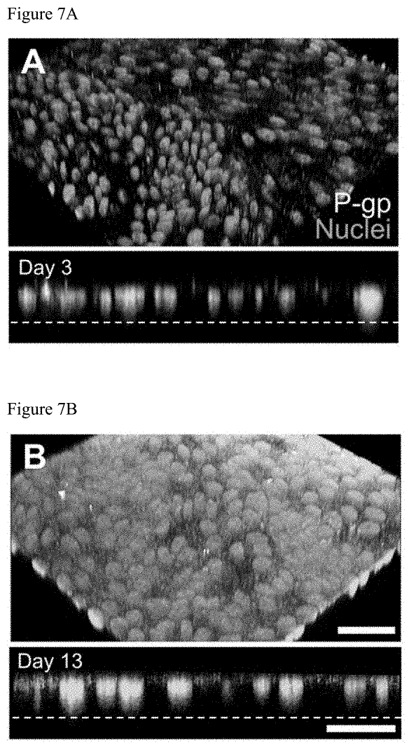

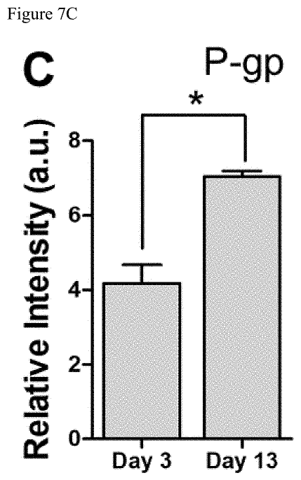

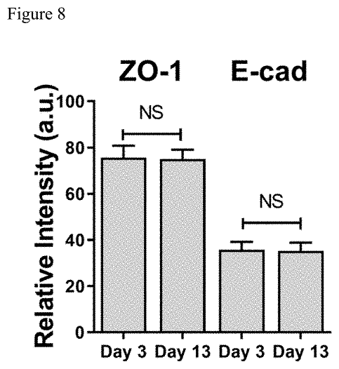

For IF microscopic analysis, a confluent cell monolayer grown on a nanoporous insert was fixed with 4% (w/v) paraformaldehyde (Electron Microscopy Science) for 15 min at room temperature. Samples were then permeabilized with 0.3% (v/v) Triton X-100 (Sigma) and blocked with 2% (w/v) bovine serum albumin (BSA; Sigma) followed by PBS (Ca²⁺ and Ma²⁺ free; Gibco) washing. The monolayer was incubated at room temperature for 1 h with primary antibodies against ZO-1 (Invitrogen), P-gp (Thermo Fisher Scientific), CgA (Abcam), and Ki67 (Abcam) diluted in 2% (w/v) BSA in PBS. Alexa Fluor 488 conjugated E-cadherin (BD Biosciences) was applied in a same procedure. Secondary antibodies of Alexa Fluor 555-conjugated goat polyclonal anti-rabbit IgG (Abcam) for ZO-1, P-gp, CgA, and Ki67 diluted in 2% (w/v) BSA in PBS were applied under light protected conditions at room temperature for 1 h. For the counterstaining, samples were incubated with 4′,6-diamidino-2-phenylindole dihydrochloride (DAPI) (1 μg/mL; Fisher Scientific) and Alexa Fluor 647-conjugated phalloidin (7.5 units; Thermo Fisher) for nuclei and F-actin visualization, respectively. To detect the mucus production on the monolayer, samples were directly stained with Alexa Fluor 488-conjugated WGA (5.0 μg/mL; Thermo Fisher). The monolayer was imaged using a differential interference contrast (DIC) or laser-scanning confocal microscopy (DMi8; Leica). Acquired images were processed using LAS X (Leica) or ImageJ v1.52 q. The percentage of cell numbers (Ki67 and CgA) or fluorescence intensity (P-gp, ZO-1, and E-cad) was assessed using ImageJ to the randomly selected images that show representative characteristics. The number of cells that show positive signals was manually counted (ImageJ), then the number was normalized by the total number of nuclei to calculate the % population. For this quantification, 3 independent fields of view from 4 independent biological replicates were used, while at least two technical replicates were performed (FIG. 6F). For the quantification of the P-gp expression, total 10 randomly chosen fields of view to detect P-gp expression levels among 4 biological replicates, while at least two technical replicates were performed (FIG. 7C). For the quantitative assessment of ZO-1 and E-cadherin, total 10 and 6 randomly chosen fields of view for ZO-1 and E-cadherin, respectively, to quantify the relative intensity of fluorescence among 4 biological replicates of IF staining experiment. We also applied two technical replicates to the individual biological replicate. (FIG. 8).

In Situ Hybridization of mRNA

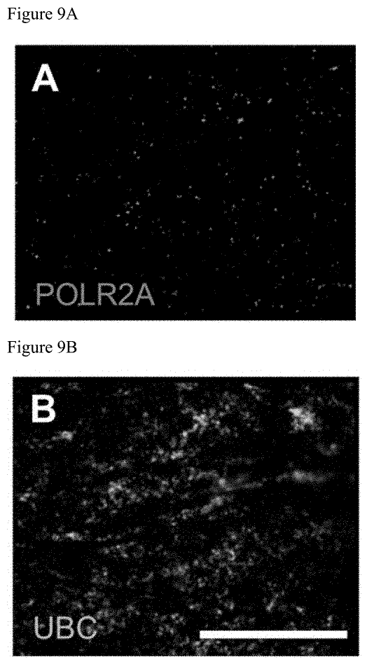

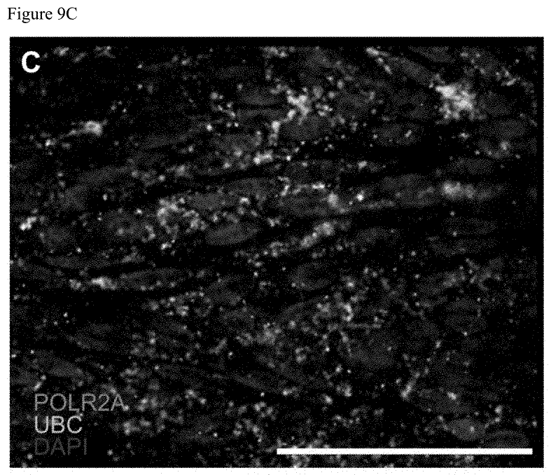

RNA-ISH using the RNAscope Multiplex Fluorescent Reagent Kit v2 (Advanced Cell Diagnostic, Newark, Calif.) on a canine colonoid-derived monolayer to characterize the multi-lineage differentiation was used for in situ hybridization. In brief, a colonoid-derived monolayer was fixed and underwent dehydration/hydration, permeabilization, and protease treatment. Samples were hybridized in the ACD HybEZ II Hybridization System (110v) oven at 40° C. while placed in light protected humidified trey as instructed by the manufacture. The samples were then stained for mRNA expression using specific oligonucleotide probes for visualizing intestinal stem cells (CL-Lgr5-C2; Advanced Cell Diagnostic), differentiated intestinal epithelial cells (Cl-ALPI; Advanced Cell Diagnostic), and secretory enteroendocrine cells (Cl-NEUROG3-C3; Advanced Cell Diagnostic), respectively. Next, amplification and visualization using Opal 520 (FP1487001KT), Opal 570 (FP1488001KT), and Opal 650 (FP1496001KT) were performed. Sections were imaged using a confocal microscope (DMi8; Leica). Acquired images were processed using LAS X (Leica) or ImageJ. The number of cells staining positive for mRNA detection for each RNAscope probe was manually counted at random positions. Specifically, the number of cells staining positive was manually counted, then normalized by the total number of nuclei. Quantification of the positive cells to individual RNA markers was performed with 3 independent fields of view from 2 independent biological replicates (FIG. 6F). Probes against RNA Polymerase II Subunit A (POLR2A) and Ubiquitin C (UBC) were applied and the same amplification and visualization steps were performed to prepare the positive control (FIGS. 9A-9C).

### Transmission and Scanning Electron Microscopy

After 13 days of culture, the culture medium was gently removed from the apical and basal chambers of the TRANSWELL®, and cells were fixed with 2% (v/v) glutaraldehyde (Electron Microscopy Sciences) in 0.1 Mcacodylate buffer (Electron Microscopy Sciences) for 1 hr at room temperature, and washed in 0.1 Mcacodylate buffer. Samples were then fixed and stained with 1% (v/v) osmium tetroxide (Electron Microscopy Sciences) and 1% (v/v) ferrocyanide in cacodylate buffer, and then stained with 2% (v/v) uranyl acetate for a negative contrast. Samples were finally dehydrated through serial dehydration in ethanol from 50% to 100% (v/v) and then infiltrated with resin (Electron Microscopy Sciences) to be polymerized at 60° C. and sectioned for TEM. Ultrathin (50-100 nm) sections were cut by a microtome with a diamond blade, then collected on copper grids and observed under the Transmission Electron Microscope (FEI Tecnai) using an accelerating voltage of 80 kV. SEM samples were fixed in 2.5% (v/v) glutaraldehyde (Electron Microscopy Sciences), treated with 1% (v/v) osmium tetroxide (Electron Microscopy Sciences) in 0.1 Msodium cacodylate buffer (Electron Microscopy Sciences) for 30 min at room temperature. Samples were dehydrated through serial dehydration in ethanol from 50% to 100%, and hexamethyldisilazane (HDMS) method. Samples were coated with a thin (12 nm) layer of Pt/Pd using a sputter coater (Cressington 208 Benchtop Sputter) prior to imaging using an SEM (Zeiss Supra 40V SEM) with an accelerating voltage of 5 kV. The average frequencies of microvilli in less frequent and frequent areas were performed at 4 random independent positions from 3 different SEM images.

Statistical Analysis

All results are expressed as mean±standard error (SEM). Shapiro-Wilk tests were used to assess the normality of the data. Mann-Whitney U test (for non-parametric data) or student's t-tests (for parametric data) were used to compare the expression levels of proteins between two different time points (Day 3 vs. Day 13), TEER and Pₐₚₚ values on different culture time points (Day 2 vs. Day 6), or TEER values in different culture conditions (proliferation medium vs. differentiation medium) at each culture time point. All statistical analyses were performed using Prism 8.2.1 (GraphPad Software, San Diego, Calif.). P values <0.05 were considered statistically significant.

Results

### Recreating a Canine Colonoid-Derived Intestinal Tissue Interface

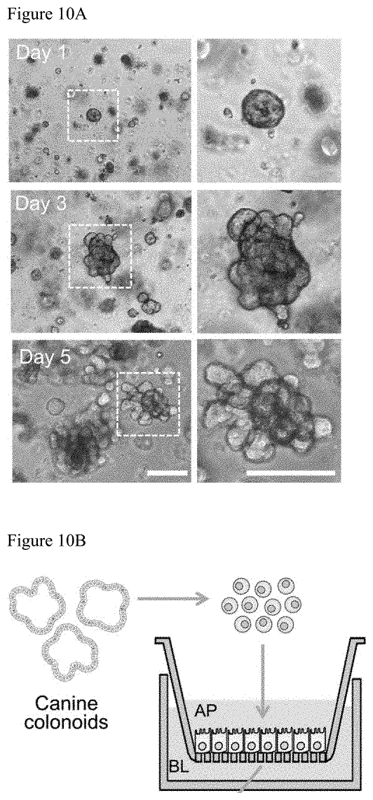

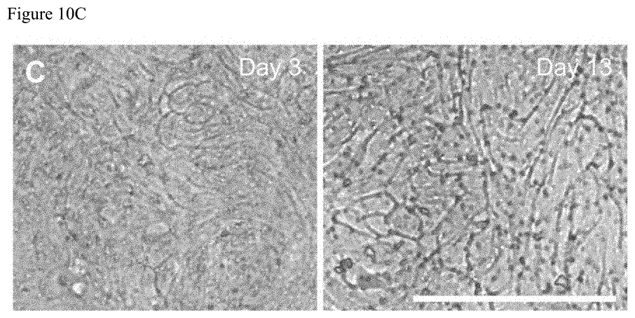

Canine colonic organoids derived from three independent canine donors were expanded in 3D geometry for up seven days in MATRIGEL® (FIG. 10A), allowing a long-term culture and storage of the primary intestinal epithelium. A colonoid-derived, two-dimensional monolayer was generated in a nanoporous insert of the TRANSWELL® pre-coated with the extracellular matrix (ECM) mix with MATRIGEL® (100 μg/mL) and collagen I (30 μg/mL) by introducing the dissociated colonoid cells (FIG. 10B). In terms of the colonoid dissociation, we employed an enzymatic dissociation method to generate single-cell suspension to accomplish a confluent monolayer, which can be maintained for at least 13 days (FIG. 10C).

### Apical Microvilli Formation in the Canine Colonic Epithelial Monolayer

The polarization of the colonic epithelium is critical to establish a biological tissue interface. Microvilli that illustrate the polarized apical membrane of the colonic epithelium were observed on the recreated monolayer using scanning electron microscopy (SEM;

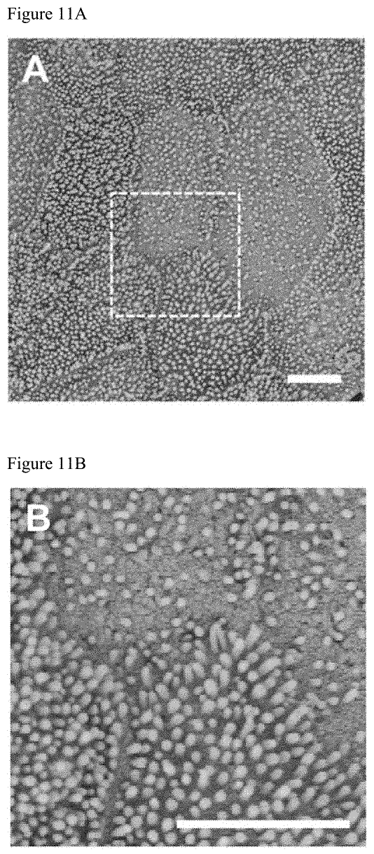

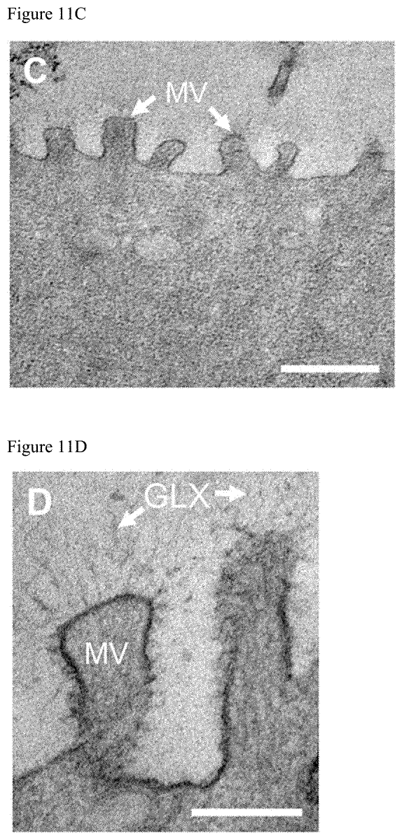

FIGS. 11A and 11B) and transmission electron microscopy (TEM; FIGS. 11C and 11D). A variation in microvilli frequency was observed in the dog colonoid-derived monolayer, which was also noted in other colonoid-derived studies. The number of microvilli assessed by the SEM imaging was variable in the range from 9 to 18 microvilli/μm2, which was similar to the reports of human intestinal epithelial cell culture performed in vitro. Glycocalyx, which provides a physical glycosylated barrier on the epithelial cells, was also well generated at the surface of the microvilli (FIG. 11D).

### Lineage-Dependent Characterization of the Differentiated Canine Colonic Epithelial Monolayer

RNA in situ hybridization (RNA-ISH), immunofluorescence (IF), and electron microscopic imaging were used to show the differentiated cell lineages in the canine colonoid-derived monolayer. The leucine-rich repeat-containing G-protein coupled receptor 5 (Lgr5), a seminal marker for adult intestinal stem cells, was detected sporadically in the 2D monolayer cultured for 14 days (FIG. 6A). Also, the canine colonic epithelium retained a population of proliferative cells, as visualized by Ki67-positive signals for up to 2 weeks (FIG. 6B). The differentiated absorptive enterocytes were visualized by the staining with intestinal alkaline phosphatase (ALPI) (FIG. 6C). The enteroendocrine cells were highlighted using Neurogenin 3 (Neurog3; FIG. 6D.) and Chromogranin A markers (CgA; FIG. 6E), respectively. In the canine epithelial monolayer, we analyzed the appearance of each cell type based on the imaging results, where the Lgr5+ stem cells, Ki67+ proliferating cells, ALPI+ differentiated intestinal epithelium, Neurog3+ and CgA+ enteroendocrine cells were populated as 7.6±0.1, 38.4±2.4, 60.1±0.9, 41.2±10.3%, and 47.8±2.7%, respectively (FIG. 6F).

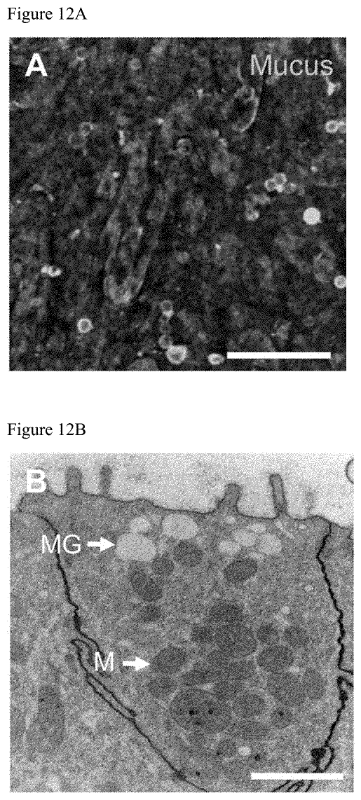

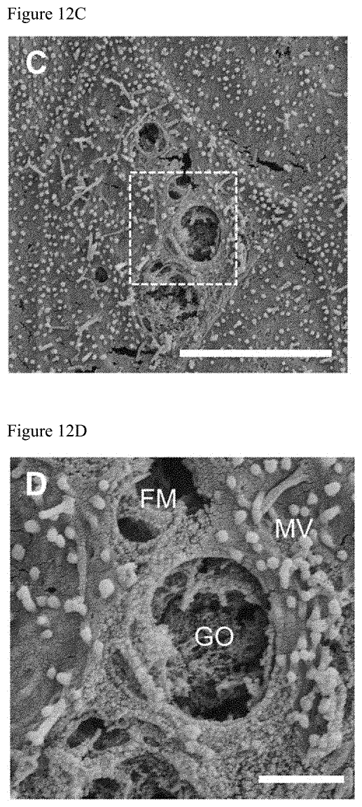

To investigate the presence of physiological mucus production in the monolayer, live-cell staining with Wheat Germ Agglutinin (WGA) was performed. It was found that the WGA-positive signals were detected across the entire monolayer, suggesting that the epithelial apical surface was covered by mucus-like molecules such as N-acetyl-D-glucosamine (FIG. 12A). It was also identified that the mucin granule-containing goblet cells using TEM (FIG. 12A, “MG”), where the goblet cell orifices (FIG. 12A, “GO”) and fenestrated membranes (FIG. 12D, “FM”) extending deep into the goblet cell were also confirmed using SEM, as shown in previous studies, demonstrating that the goblet cells were present in the canine colonoid-derived monolayer.

In addition, it was confirmed that the P-glycoprotein (P-gp) efflux transporters were diffusely expressed on the apical surface of the canine colonoid-derived monolayer (FIGS. 7A and 7B), which is consistent with the localization of the P-gp transporters in the canine colonic tissue. Importantly, the IF assessment revealed that the polarized expression of P-gp was significantly (P<0.0001) increased on Day 13 compared to the images acquired on Day 3 on the nanoporous insert, suggesting that the maturity of the colonoid-derived epithelial monolayer was achieved (FIG. 7C).

### Assessment of the Canine Intestinal Barrier Integrity

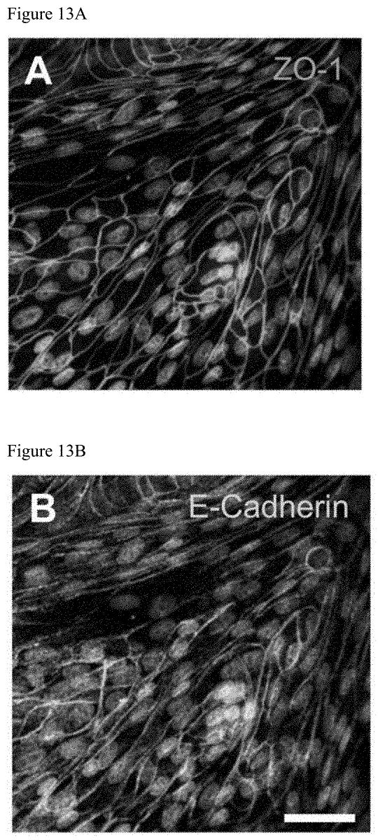

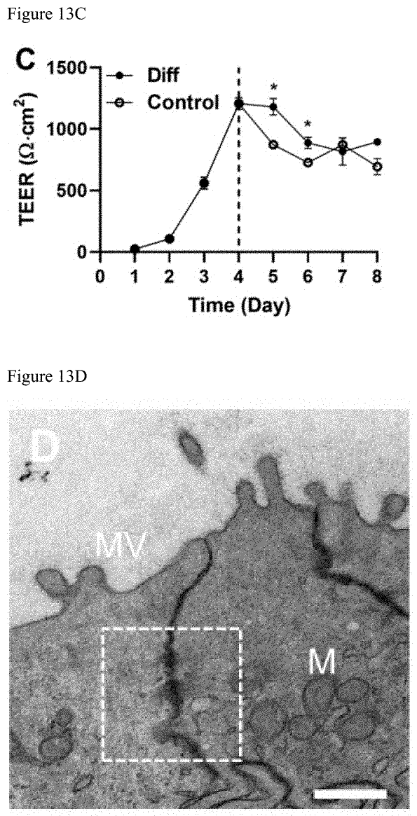

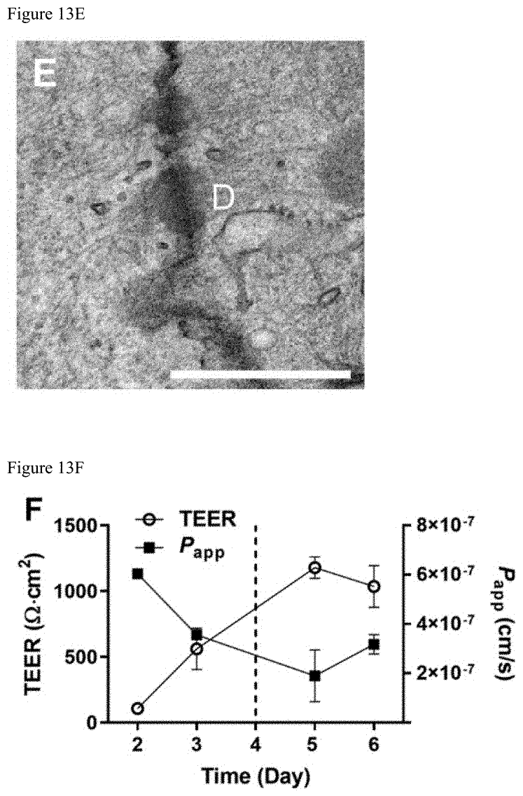

The formation of tight junction proteins was confirmed by IF staining for zonula occludens 1 (ZO-1) (FIG. 13A) and E-cadherin (E-cad) expression (FIG. 13B), where no significant difference of the expression at Day 3 and 13 was observed in both ZO-1 and E-cad (FIG. 8). After 4 days of cultures, the confluent colonoid monolayer showed stable transepithelial electrical resistance (TEER) values of approximately 1,000 Ωcm² (FIG. 13C). We observed that the TEER value was stably maintained for up to 14 days when the culture medium was replenished every other day for all 3 independent lines of canine colonoid-derived epithelium (FIG. 5).

Next, we evaluated the effect of the complete medium with or without Wnt proteins on the growth of canine colonoid-derived monolayer to verify the effect of differentiated culture condition on the epithelial barrier function. Briefly, the overall profile of TEER cultured in both the differentiation (i.e., the Wnt-free and Wnt-containing medium in the apical and basolateral compartment, respectively; FIG. 13C, “Diff”) and proliferation medium (i.e., Wnt-containing medium to both compartments; FIG. 13C, “Control”) showed a similar decline as a function of time. However, the monolayer conditioned under the differentiation medium showed a temporal maintenance of the TEER for days compared to the control (P<0.01). The effect of different culture medium on the TEER values became negligible over time by Day 7 (FIG. 13C). This observation is consistent with the previous findings from our group where low Wnt3a-containing medium (i.e., differentiation medium) was not necessary for the development of mature canine tight junctions. The TEM images revealed the presence of intercellular junctions as well as desmosomes at Day 13 (FIGS. 13D and 13E). Corresponding apparent paracellular permeability (Pₐₚₚ) to fluorescein sodium salt (Mw, 376.27 Da) was measured, and an inverted relationship of TEER and Pₐₚₚ values was observed (FIG. 13F). Specifically, as TEER values significantly increased from Day 2 to Day 6 (P<0.0001), corresponding Pₐₚₚ values significantly decreased (P<0.0001), supporting that the TEER value may be used to predict the appropriate point to perform epithelial-luminal interactions.

### Discussion

In this study, it was shown for the first time the development of an optimized method for the generation of an intact canine colonoid-derived monolayer from canine 3D colonoids. The enzymatic dissociation method can be applied to canine organoids as performed in other species to generate single-cell suspension to accomplish a confluent monolayer. The multimodal imaging techniques employed in this study confirmed the creation and stable maintenance of the 2D canine intestinal epithelial monolayer on a nanoporous insert up to two weeks with a physiological expression of structural tight-junctions and marker proteins.

Findings from TEM and SEM micrographs demonstrated the formation of a physiological brush border interface and the presence of glycocalyx on the microvilli, which is the characteristic of terminally differentiated canine intestinal epithelium. It was confirmed that the canine epithelium cultured on a nanoporous insert grew into multiple lineages of the differentiated intestinal epithelium including absorptive enterocytes, goblet cells, and enteroendocrine cells. Furthermore, the IF imaging data confirmed that P-gp efflux proteins were apically expressed similarly to canine colonic tissue in vivo.

It was also confirmed that stable TEER values could be established by Day 4 of the monolayer culture, which is similar to the previous study using canine or human cell lines. The TEER values increased as a concurrent decrease in the apparent permeability of a paracellular marker similar to the previous study, suggesting that the ideal timeline to perform the barrier-associated experiments can be estimated once stable TEER values are achieved (here, after Day 4). In human intestinal organoid culture, Wnt protein-rich medium produced largely undifferentiated progenitors due to the central role that Wnt signaling plays in the maintenance of an undifferentiated crypt progenitor state. The minimal effect of low Wnt3a-containing medium (i.e., differentiation medium) for the development and maintenance of mature canine tight junctions was also demonstrated as reported previously in Chandra et al.

Moreover, it was shown that the canine colonoid on the TRANSWELL® contain a stable population of the intestinal stem cells as well as other differentiated cells present in the intestinal tissue of origin. Using RNA-ISH imaging technology, it was shown that it is possible to investigate the percentage of cells expressing multi-lineage cell differentiation RNA markers, including the Lgr5+ stem cells, ALPI+ differentiated intestinal epithelium, Neurog3+ enteroendocrine cells, which were all similar to what have been previously reported in human and dog in vitro intestinal systems. It is noted that the Ki67+ cells are not the population of lineage-dependent cells; however, we included in the same chart (FIG.) to provide a quantitative information. It is also critical to confirm the production of intestinal mucus and a glycocalyx on the engineered epithelial monolayer. We demonstrated the presence of mucus with WGA staining and the presence of glycocalyx using TEM imaging. The presence of goblet cells was also demonstrated using TEM and SEM by detecting multiple mucin granules (MG) (FIG.) and goblet cell orifices (GO) as well as a fenestrated membrane (FM) (FIG.) as shown in previous studies.

A key advantage of the creation of a 2D mucosal tissue interface is that this culture format will allow easier access to the apical side of the epithelium for investigating the nutrient and drug absorption, host-microbe crosstalk, or drug metabolism and toxicity testing. The 2D mucosal tissue interface using primary 3D intestinal organoids will allow modeling of intestinal physiology ex vivo or in vitro compared to currently available canine-specific immortalized cell lines. The measurement of the epithelial barrier function (e.g., TEER) is convenient when investigating the physiological responses of epithelial cells following exposure to toxins, therapeutic drugs, or nutrients.

Although dogs are excellent animal models to study human diseases, dog studies are often limited by the number of commercially available reagents targeting major proteins shown to be relevant in mice. The RNA-ISH technology provides an in situ analysis of biomarkers within the histopathological context of biological samples as they target the mRNA of select proteins. RNA-ISH is a suitable alternative to IF in those cases where the detection of proteins lacks sensitivity or cellular resolution. The customized probes for RNA-ISH can be engineered based on any RNA sequences, which allows investigators to overcome the lack of canine-specific reagents for the identification of intestinal stem cells and their lineage cells in dogs. However, as RNA-ISH only detects mRNA expression, it provides no spatial information on actual protein expression or matured protein productive function in the cell. Regardless of the location of the positive signal, a positive signal is an indicative of the presence of the target gene(s) in that particular cell. This RNA-ISH technology has been successfully applied in dog organoids by our group and similar findings (i.e., positive signals seem to be expressed in the nucleus) can be found in other studies as well as the positive control provided in FIG. 9.

Stunted microvilli were observed in the system which could reflect the fact that colonic intestinal cells may not require longer microvilli due to minimal nutrient absorption in the colon. Possibly, it could be due to the culture condition that is not completely adequate to promote longer microvilli. As described before, Wnt-enriched medium produced largely undifferentiated progenitors comprising organoids in human intestinal organoid culture. This work and the work in other Examples herein demonstrate that canine intestinal organoids are indeed capable of differentiating into functional epithelial cells even under Wnt-enriched condition; however, the effect of low Wnt-containing medium (i.e., differentiation medium) particularly on microvilli length would be beneficial to better understand the physiological demonstration and functions of the microvilli in the future study.

This Example demonstrates the methods to create the accessible apical surface of the intestinal epithelium generated from canine colonoids. Moreover, the method developed herein can be applied to other segments of organoids (i.e., enteroids) as well as the organoids obtained from both diseased and other healthy dogs to enable segmental investigation of epithelial-luminal interactions.

### Example 5

Urothelial carcinoma (UC) is the most common type of bladder cancer in both dogs and humans. UC is incurable with minimal treatment success due to tumor heterogeneity and frequency of distant metastases at the time of diagnosis. Dogs function as physiologically relevant models for UC in humans due to similarities in genetic predispositions, environmental risk factors, clinical presentation, responsiveness to common chemotherapeutics, and tumor molecular and behavioral phenotypes.

The stem cell-derived organoid cultures disclosed herein show an increasing value to reverse translational clinical research and personalized medicine.

This Example shows the culture and characterize UC organoids from urine collected from a canine clinical patient and characterize UC organoids based on shared histology and molecular markers of UC. Further, we aimed at developing assays for drug screening of chemotherapy to be used for precision-medicine purposes both in veterinary and human patients suffering from UC.

Free-catch urine was collected from one dog at time of UC diagnosis. Samples were centrifuged and supernatant was removed. The resulting pellet was washed with phosphate-buffered saline (PBS), then incubated in complete chelating solution (CCS) with EDTA and plated in MATRIGEL® for establishment of organoid culture within one week. Sub-samples of differentiated UC organoids were taken for 3-(4,5-dimethylthiazol-2-yl)-2,5-diphenyltetrazolium bromide (MTT) assay for metabolic activity assessment after incubation with chemotherapeutic agents. Remaining UC organoids were characterized with H&E, RNA in-situ hybridization (RNA-ISH), and immunohistochemistry (IHC) staining techniques.

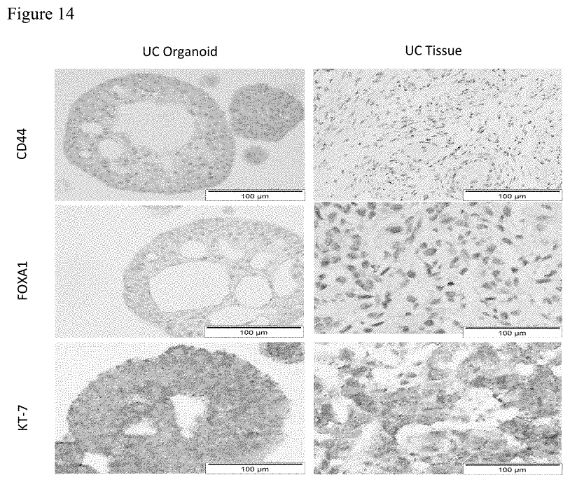

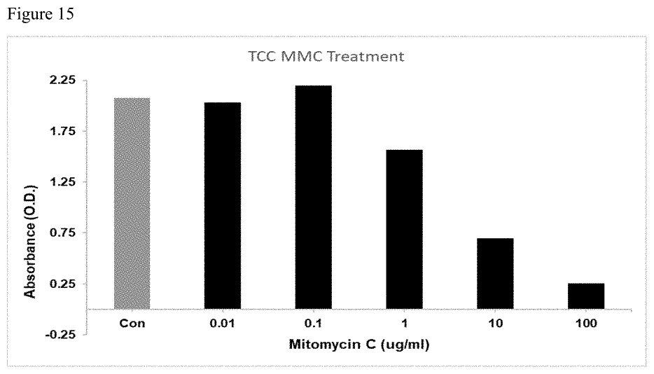

Differentiated organoids showed structural similarity to UC tumor epithelium on H&E staining (FIG. 14). RNA-ISH showed high expression of Keratin-7 (KT-7, a marker specific for urothelial epithelium) in UC organoids. Ki-67 (epithelial proliferation marker), vimentin (marker unregulated in metastatic UC) and CD44 (presumptive urothelial stem cell marker) were overexpressed in canine UC organoids, consistent with in vivo canine UC tissue and human muscle invasive bladder cancer tissue and organoids. Results from MTT assay on maintained, differentiated canine UC cultures demonstrated reduced metabolic activity of UC organoids after incubation with cisplatin for 24-48 hours (FIG. 15).

These results show that urine-derived canine UC organoids share histological and molecular similarities to UC tissue in vivo. In addition, we show proof-of-concept for a precision-medicine test using cisplatin on canine UC organoids.

Collectively, these results show the potential value of the organoid technology for characterization of UC phenotype and treatment responsiveness as an emerging tool for personalized medicine applications in veterinary and human medicine.

### Example 6

### Rationale and Significance

Canine inflammatory bowel disease (IBD) refers to a group of chronic gastrointestinal (GI) disorders of unknown cause and pathogenesis which mimic the spectrum of chronic enteropathies in human patients with IBD. There is currently no cure for IBD and many therapeutic interventions developed over the past 3 decades have consistently failed to demonstrate evidence of efficacy in clinical trials. There is, therefore, a critical need to develop robust drug screening tools to accelerate the availability of effective and safe therapeutic strategies for management of IBD. Intestinal stem cell (ISC)-derived organoids, which model spontaneous GI disease in dogs and humans, can be used as an ex vivo tool in the study of IBD pathogenesis. The canine organoids disclosed herein faithfully reproduce structural and functional changes of the intestinal epithelium in dogs with IBD, which enable more accurate prediction of therapeutic drug efficacy and safety. Long-term, this model will provide critical information for the design of canine clinical trials, and ultimately generate preclinical data for similar studies in humans with IBD. Altogether, the organoids of this disclosure can be used to contribute to decreased morbidity/mortality, and improved quality of life in dogs and patients with IBD.

### Methods

ISCs isolated from endoscopic biopsies of two healthy dogs and two dogs with active IBD were differentiated into intestinal organoids. Ileal organoids and matching tissues were probed by RNA in situ hybridization and immunohistochemistry for phenotypic changes in IBD. A panel of six phenotypic markers identified different epithelial cell lineages (LGRS+: intestinal stem cell, ALP: enterocyte, PAS: goblet cell, NeuroG3: enteroendocrine cell), epithelial barrier integrity (ZO-1) and cell proliferation (Ki-67). Functional features of IBD organoids were investigated by cystic fibrosis transmembrane conductance regulator (CFTR) organoid swelling assay to measure Cl-channel-water conductance.

For RNA-ISH, RNAscope visualizes mRNA within paraffin-embedded tissue by hybridizing and amplifying mRNA with a canine-specific probe targeting LGRS+, ALP, and NeuroG3.

For IHC, Ki-67, ZO-1, and PAS markers were visualized by deparaffinizing, rehydrating, retrieving antigen, blocking, and incubating with primary antibodies and conjugated secondary antibodies.

For CTFR swelling, average areas and images were taken from enteroids which were passaged (24 well plates), seeded in MATRIGEL®, and incubated (2 days later) in CMGF+ medium with Forskolin (CFTR potentiator).

Mean and standard deviation were calculated from multiple independent measurements. Two sample t-tests and multiple pairwise comparisons were performed to determine group differences. Data were analyzed using R version 3.5. Statistical significance level was set at P<0.05.

### Results

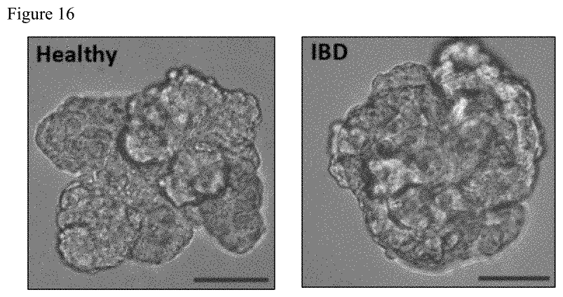

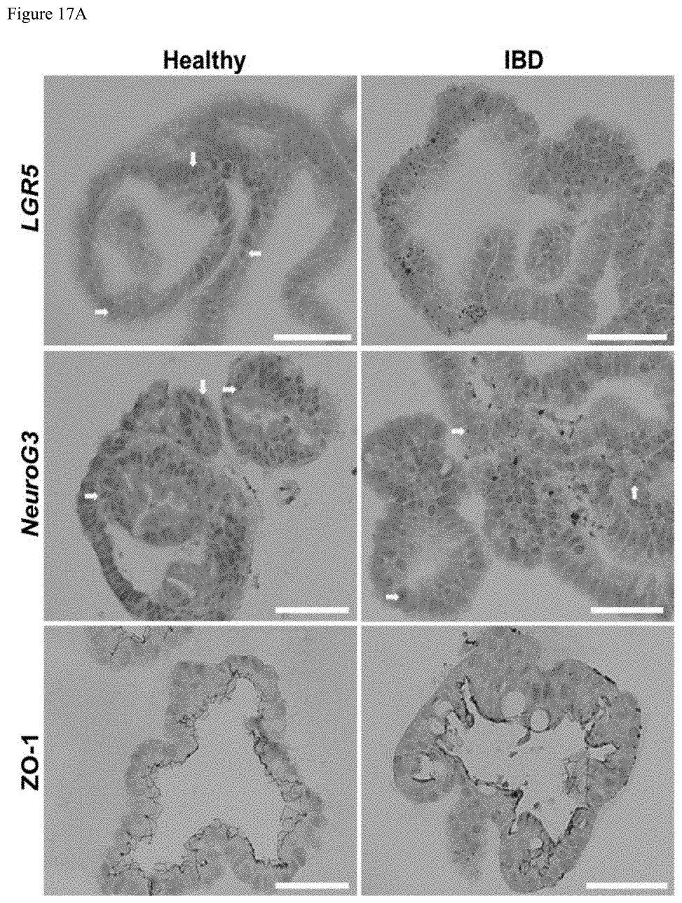

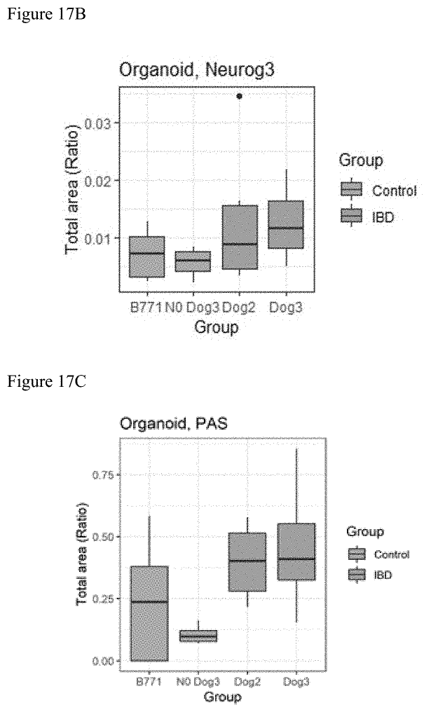

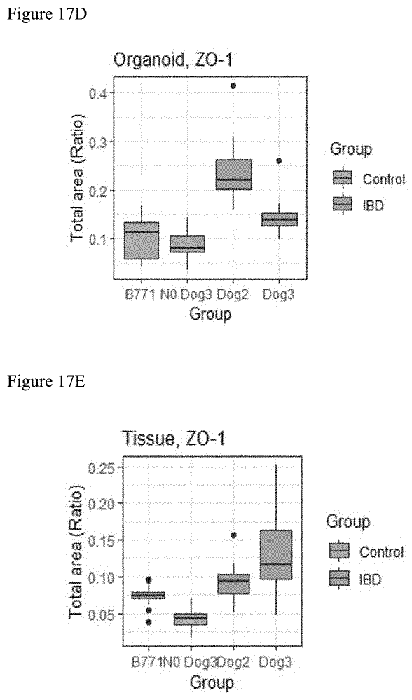

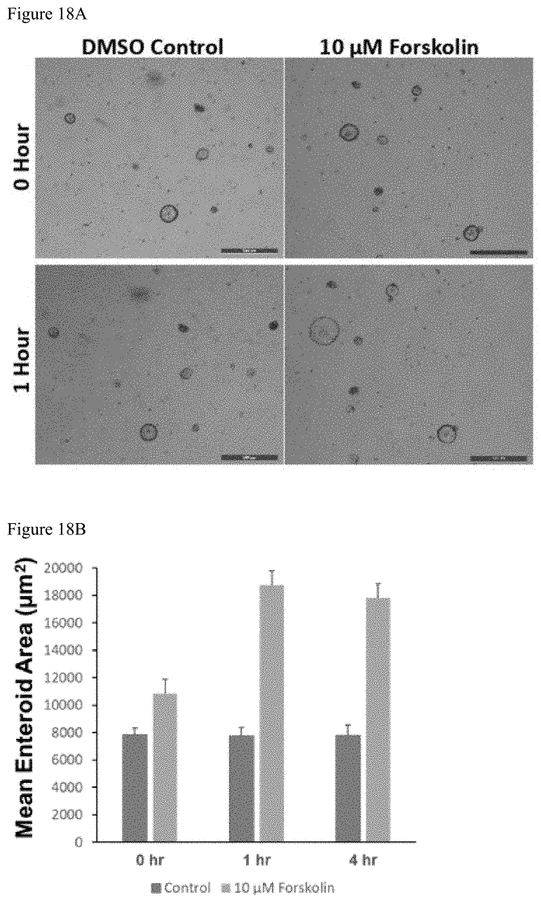

Whole tissues exhibited inflammation-mediated changes in ALP, LGRS+, NeuroG3, Ki-67, ZO-1 as anticipated (Table 2 and FIGS. 17A-17E). Significant differences in expression of phenotypic markers NeuroG3 and PAS were observed between healthy and IBD organoids (p<0.05). Similar trends in expression of LGRS+, NeuroG3, and ZO-1 were observed between inflamed whole tissues versus IBD organoids. Swelling assay (CFTR) showed that IBD organoids have functional CFTR-Cl-channels but behave differently from healthy organoids (FIGS. 16, 18A, and 18B).

TABLE 2
Means of biomarkers expressed in healthy or IBD enteroids and
tissues, including LGR5+ (intestinal stem cell), ALP (enterocyte),
NeuroG3 (enteroendocrine cell), ZO-1 (epithelial barrier integrity),
Ki-67 (cell proliferation), and PAS (goblet cell).

| | | | | | |
|---|---|---|---|---|---|
| ALP | LGR5+ | NeuroG3 | Ki-67 | ZO-1 | PAS |

Enteroids

| | | | | | | |
|---|---|---|---|---|---|---|
| B771 | 0.072 | 0.019 | 0.007 | 2.195 | 0.102 | 0.238 |
| N0 Dog3 | 0.064 | 0.024 | 0.006 | 0.731 | 0.088 | 0.171 |
| SG Dog2 | 0.096 | 0.02 | 0.011 | 0.972 | 0.239 | 0.4 |
| SG Dog3 | 0.068 | 0.036 | 0.012 | 0.964 | 0.144 | 0.439 |

Tissue

| | | | | | | |
|---|---|---|---|---|---|---|
| B771 | 1.244 | 0.045 | 0.004 | 0.152 | 0.073 | 0.342 |
| N0 Dog3 | 0.897 | 0.009 | 0.001 | 0.091 | 0.042 | 0.131 |
| SG Dog2 | 0.653 | 0.094 | 0.01 | 0.32 | 0.092 | 0.273 |
| SG Dog3 | 0.634 | 0.071 | 0.003 | 0.559 | 0.131 | 0.125 |

### Conclusion

Overall, the data showed that ileal organoids derived from dogs with IBD recapitulate both the phenotypic and physiological features of diseased tissue compared to healthy tissue, demonstrating its utility as an ex vivo model for investigating mechanism and therapeutic strategies in IBD. These preliminary results will be further validated by additional intestinal biopsies from 14 dogs with IBD in an ongoing clinical trials.

### Example 7

The organoids may also be used to determine drug toxicity to both normal tissues and to tumors within a subject. By comparing an individuals diseased tissue with normal tissue, by taking samples of either, it is possible to determine the state between the two and then administer a compound, such as a drug or a dietary compound, and then measure the differences between the tissues for differences in metabolism, toxicity, uptakes, and other properties. The differences between the normal and diseased state can help identify an individual's specific toxicity or treatment level for diseases such as cancer.

### Materials and Methods

Transitional Cell Carcinoma (TCC) Organoid Isolation and Culture. Canine bladder cancer cells for TCC organoids were isolated from urine (TCC #1) or biopsies (TCC #2) of dogs with transitional cell carcinoma. Briefly, urine was centrifuged and washed or small biopsies were washed with 1×Complete Chelation Solution (CCS) and vortexing up to 6 times, then incubated in 20-30 mM EDTA for 1 hour at 4° C. on a rocker. FBS and CCS was added to stop EDTA chelation, mixed, and the supernatant containing cancer cells was put in a new tube. Samples were centrifuged at 150 g for 5 minutes at 4° C., resuspended in DMEM/F12, centrifuged again, and the pellet was resuspended in 120 ul MATRIGEL®. MATRIGEL® droplets were then plated at 30 ul/well MATRIGEL®/cells on 24-well plates. Complete Media with Growth Factors (CMGF+) containing Rock inhibitor and GSK inhibitor was added for 2-3 days at 37 C, and after 2-3 days, TCC organoids were cultured in CMGF+ media without inhibitors. TCC organoids were passaged every 4-7 days with TrypLE Express to dissociate organoids to single cells.

MTT Cell Viability Assay (Cytotoxicity Assay). Organoids from healthy intestine or transitional cell carcinoma (TCC) were dissociated and plated at equal density in 30 ul/well MATRIGEL® in 24-well plates in 500 ul/well CMGF+ growth medium. On Day 1-4 after passage, organoids were incubated with the indicated drug for 48 or 96 hours. Cytotoxicity was determined using 3-(4,5-dimethylthiazol-2-yl) 2,5-diphenyl tetrazolium bromide (MTT) at a final concentration of 0.5 mg/mL for 1.5 hr. After medium removal, 200 ul/well cold DMSO was used to dissolve the formazan dye crystals and absorbance was read at 570 nm using a plate reader (SpectraMax 190, Molecular Devices).

As DMSO freezes above 4° C. but it is preferable to use cold media (4° C.) to dissolve the MATRIGEL®. Thus, put DMSO briefly in 4° C. fridge or −20° C. freezer and remove before it all freezes, then use cold DMSO, pipette up and down vigorously, shake the plate, put plate in freezer briefly, etc. to try to get the MATRIGEL® and formazan dye crystals to dissolve. It does not work for dissolve MATRIGEL® in small amount of cold PBS and then add DMSO because purple formazan crystals will not dissolve then.

### Results Summary

Mitomycin C (MMC) reduces cell viability in both TCC organoids and healthy colon or ileum organoids to about the same extent at the same doses (at 1, 10, 100, or 250 ug/ml MMC) after incubation for 48 hours (lower doses of 0.01 or 0.1 ug/ml MMC are not cytotoxic). 10 ug/ml reduces cell viability, as determined by MTT assay, to about 25-30% of control, and 100 ug/ml reduces cell viability to about 10% of control. This was done with a higher dose curve of MMC (10-750 ug/ml) and later repeated with a lower dose curve.

Nanoparticles (MSN) carrying 25 ug/ml MMC reduced TCC viability to about 80% of MSN nanoparticles alone after 48 hours (probably not all the MSN with MMC stayed with the organoids after centrifuging again and plating in MATRIGEL® droplets). Incubation with Piroxicam (1-10 μM) for 48 hours or 4 days at most reduced cell viability to 80% of control (20% decrease), by MTT assay. This is with Piroxicam from tablet ground up in our pharmacy or with Piroxicam from Selleck Chemicals. Piroxicam decreased Ki-67 staining 50% at 0.1 μM Piroxicam, but increased Ki-67 IHC staining at 1-10 μM Piroxicam about 2-3 fold.

Incubation with Doxorubicin decreased TCC organoid cell viability (MTT) to 50% of control (10 μM doxorubicin), but sometimes had only a small 20% effect or no effect. Doxorubicin did decrease Ki-67 IHC (proliferation) to 10-20% of control at 0.1 μM Dox once (VDL is finishing Ki-67 IHC staining).

Epacadostat (IDO-1 inhibitor) decreased TCC cell viability (MTT assay) to about 80% of control at 1 μM Epacadostat after 48 hours, but not at the other doses. 1-10 μM Erdafitinib (FGF receptor inhibitor) had no effect and (on the other plate) Vinblastine and E-7046 (EP4R antagonist) had a slight stimulatory effect. However, the TCC organoids had been passaged a lot (were old) and there were cells growing outside of the MATRIGEL® on the plastic, so take these results with a grain of salt, and they will need to be repeated with healthier TCC organoids.

Dopamine (0.1, 1, 10 μM) increased Ki-67 IHC staining about 2-fold for TCC organoids, which 1 nM Fenoldopam (dopamine-type 1 receptor agonist), decreased TCC organoid Ki-67 (proliferation) about 50% (10 μM Fenoldopam did not have much effect, but it could be signaling through other dopamine receptors at higher concentrations). Also, it looked like the lumens/holes in the TCC organoids were getting bigger in the fenoldopam TCC organoids.

Untreated TCC1 organoids had positive staining for vimentin (IHC), Ki-67 (IHC), very weakly positive staining for EP4R (RNAscope), and negative staining for ZO-1 (IHC), with TCC tissue from different dog.

### Conclusion

The results show that there are important differences between subjects, for example the difference in staining in TCC in different animals, which may be assessed using the organoids of the disclosure. The results also show that TCC cells show different responses to compounds, such as dopamine, as well when compared to control organoids. Therefore, the organoids of the present disclosure may be used to assess differences both within a subject and across subjects to allow for their use as a model for personalized medicine and drug efficacy and safety.

### Example 8

This Examples shows that the recovery rate of organoids that had been frozen in liquid nitrogen using the commercial Cell Culture Freezing media or the preferred freezing media disclosed herein made of 50% Complete Media with Growth factors (CMGF+)/40% FBS/10% DMSO results in a different revival rates.

### Materials and Methods

Organoids were grown from Ileal biopsies taken from 6 healthy beagles (D1, D3, D4, D5, D7, and D8). The organoids were taken during 2 separate time periods and during different treatment experiments.

Biopsies were taken from the 6 animals after a high fat nutrition study treatment and grown for 6-9 days before being suspended in 750 μl of Invitrogen Cell Culture Freezing Media in 1.2 ml cryovials. These organoids were then stored in the vapor phase in liquid nitrogen for approximately 1 yr and 8 months before attempted revival.

Biopsies from the 6 animals were then taken post treatment for an experiment looking at L-dopa expression. The organoids were grown for 4-5 days, passaged, and then cleaned per the preferred methods using the preferred media disclosed herein. They were then suspended in 500 μl 50% CMGF+/40% FBS/10% DMSO freezing media and stored in vapor phase liquid nitrogen for approximately 7 months using freezing protocol disclosed herein.

One vial of the frozen organoids were used for the revival experiment. They were revived using the revival protocol disclosed herein.

### Results

All 6, 100%, of the post L-Dopa treatment animals that were frozen using the 50% CMGF+/40% FBS/10% DMSO showed new organoid growth within 2-4 days of revival. Five of the 6, 83%, Nutrition Study animals that were frozen using the Cell freezing media showed new organoid growth within 7 days, with the exception of Dog 7 (Santorini; Tables 3 and 4).

TABLE 3
Position of the organoids in the 24 well plate and approximate number of organoids
present using commercial freezing media. The organoid numbers include those that
were not viable.

| | | | | | |
|---|---|---|---|---|---|
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |

TABLE 4
Position of the organoids in the 24 well plate and approximate number of organoids
present using the preferred freezing media disclosed herein. The organoid numbers
include those that were not viable.

| | | | | | |
|---|---|---|---|---|---|
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |
| D1 Ileum | D3 Ileum | D4 Ileum | D5 Ileum | D7 Ileum | D8 Ileum |
| ~75 | ~50 | ~75 | ~50 | ~50 | ~50 |
| organoids | organoids | organoids | organoids | organoids | organoids |

### Discussion

The results show that the freezing media and the protocol used to freeze these organoids disclosed herein allow for a higher percent retrieval rate and faster revival growth than that of the Cell freezing media from Invitrogen. The results show that the media and methods disclosed herein allows for quicker revival than the commercial freezing media.

The animals that used the freezing media disclosed herein were passaged and cleaned before freezing which allowed for more organoids to be frozen per cryovial and removal of cell debris that could hinder growth. The organoids that used the Cell freezing media were frozen without these steps.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A canine epithelial organoid, comprising:
a population of differentiated canine epithelial-derived cells which are capable of organ-like functionality.

2. The epithelial organoid of claim 1, wherein the epithelial-derived cells are adult stem-cell derived cells.

3. The epithelial organoid of claim 1, wherein the epithelial-derived cells are induced pluripotent derived stem cells.

4. The epithelial organoid of claim 1, wherein the epithelial-derived cells are derived from the gastrointestinal tract.

5. The epithelial organoid of claim 4, wherein the gastrointestinal tract derived cells are from the small intestine.

6. The epithelial organoid of claim 5, wherein the cells derived from the small intestine are from the duodenum, ilium, or jejunum.

7. The epithelial organoid of claim 4, wherein the cells derived from the gastrointestinal tract are derived from the colon.

8. The epithelial organoid of claim 1, further comprising an extracellular matrix, wherein the epithelial organoid maintains the organ's three-dimensional structures.

9. The epithelial organoid of claim 8, wherein the epithelial-derived cells are derived from the gastrointestinal tract and maintain the expression of tight junction proteins.

10. The epithelial organoid of claim 8, wherein the epithelial-derived cells maintain the expression of P-glycoprotein.

11. The epithelial organoid of claim 1, wherein the epithelial-derived cells are diseased.

12. The epithelial organoid of claim 11, wherein the disease is cancer.

13. The epithelial organoid of claim 11, wherein the disease is inflammatory bowel disease.

14. A culture media for differentiating stem cells into organoids, comprising:
a complete media; and
a growth factor, wherein the growth factor differentiates the stem cell into the organoid.

15. The culture media of claim 14, wherein the growth factor is epidermal growth factor, Noggin, R-spondin-1, wingless-type MMTV integration site family member 3A, Gastrin, Nicotinamide, a transforming growth factor beta receptor I inhibitor, a mitogen activated protein kinase 14 inhibitor, and/or combinations thereof.

16. The culture media of claim 14, further comprising a rho kinase inhibitor and/or a glycogen synthase kinase 3 inhibitor.

17. The culture media of claim 16, wherein the rho kinase inhibitor is Y27632, Y39983, Wf-536, SLx-2119, Azabenzimidazole-aminofurazans, DE-104, olefins, isoquinolines, indazoles, pyridinealkene derivatives, H-1152P, ROKα inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, fasudil and/or combinations thereof.

18. The culture media of claim 16, wherein the glycogen synthase kinase 3 inhibitor is an aminopyrimidine.

19. A canine epithelial organoid culture system, comprising:
a three dimensional canine epithelial organoid, comprising:
a population of differentiated canine epithelial-derived cells which are capable of organ-like functionality;
an extracellular matrix; and
a culture media for differentiating stem cells into organoids, comprising:
a complete media; and
a growth factor, wherein the growth factor differentiates the stem cell into the organoid.

20. The canine epithelial organoid culture system of claim 19, further comprising a rho kinase inhibitor and/or a glycogen synthase kinase 3 inhibitor.

21-92. (canceled)
